(19) United States
(12) Patent Application Publication
(10) Pub. No.: US 2024/0009274 A1
(43) Pub. Date: Jan. 11, 2024

(54) Use of NRG-1Beta1 for Detection and/or Treatment of Multiple Sclerosis

(71) Applicant: University of Manitoba, Winnipeg (CA)

(72) Inventors: Hardeep Kataria, Winnipeg (CA); Soheila Karimi-Abdolrezaee, Winnipeg (CA)

(21) Appl. No.: 18/043,199

(22) PCT Filed: Sep. 7, 2021
(86) PCT No.: PCT/CA2021/051229

§ 371 Date: Feb. 27, 2023

Related U.S. Application Data
(60) Provisional application No. 63/078,654, filed on Sep. 15, 2020.

(51) Int. Cl. A61K 38/18; A61K 45/06; A61P 21/00

(52) U.S. Cl. CPC A61K 38/1883; A61K 45/06; A61P 21/00

(57) ABSTRACT

Multiple sclerosis (MS) is characterized by immune mediated neurodegeneration that results in progressive, life-long neurological and cognitive impairments. Yet, the endogenous mechanisms underlying MS pathophysiology are not fully understood. Here, we provide compelling evidence that associates dysregulation of neuregulin-1 beta 1 (Nrg-1β1) with MS pathogenesis and progression. In the experimental autoimmune encephalomyelitis (EAE) model of MS, we demonstrate that Nrg-1β1 levels are abated within spinal cord lesions and peripherally in the plasma and spleen during presymptomatic, onset and progressive course of the disease. We demonstrate that plasma levels of Nrg-1β1 are also significantly reduced in individuals with early MS and is positively associated with progression to relapsing-remitting MS. The functional impact of Nrg-1β1 downregulation preceded disease onset and progression, and its systemic restoration was sufficient to delay EAE symptoms and alleviate disease burden. Intriguingly, Nrg-1β1 therapy exhibited a desirable and extended therapeutic time window of efficacy when administered prophylactically, symptomatically, acutely or chronically. Using in vivo and in vitro assessments, we identified that Nrg-1β1 treatment mediates its beneficial effects in EAE by providing a more balanced immune response. Mechanistically, Nrg-1β1 moderated monocyte infiltration at the blood-central nervous system interface by attenuating chondroitin sulfate proteoglycans and matrix metalloproteinase-9. Moreover, Nrg-1β1 fostered a regulatory and reparative phenotype in macrophages, T helper type 1 (Th1) cells and microglia in the spinal cord lesions of EAE mice. Taken together, our new findings in MS and EAE have uncovered a novel regulatory role for Nrg-1β1 early in the disease course and suggest its potential as a specific therapeutic target to ameliorate disease progression and severity.

The instant application claims the benefit of U.S. Provisional Patent Application Ser. No. 63/078,654, filed Sep. 15, 2021 and entitled “USE OF NRG-1β1 FOR DETECTION AND/OR TREATMENT OF MULTIPLE SCLEROSIS”, the entire contents of which are incorporated herein by reference for all purposes.

### BACKGROUND OF THE INVENTION

Multiple sclerosis (MS) is a complex chronic immune-mediated condition of the central nervous system (CNS) that manifests as demyelination with concomitant axonal and neuronal degeneration resulting in neurological impairment [1, 2]. Approximately 85% of MS patients present with a relapsing-remitting course of the disease (RRMS), and most of these individuals advance to secondary progressive MS (SPMS) within 15-20 years of disease manifestation [3]. Moreover, in MS disease course, clinically isolated syndrome (CIS) describes an individual who presents with a first episode of neurologic dysfunction characterized by demyelination or inflammation in the CNS consistent with an MS relapse [4]. When accompanied by brain lesions suggestive of MS on magnetic resonance imaging (MRI), CIS indicates a high probability of a subsequent diagnosis of MS [5]. Current clinical assessment of MS lacks sensitivity for early diagnosis and prediction of disease progression because it most commonly relies on MRI to detect demyelinating plaques in the CNS in conjunction with clinical presentation. This limitation mainly reflects critical knowledge gaps in our understanding of the cellular and molecular mechanisms underpinning MS pathogenesis and progression. Uncovering these endogenous mechanisms would allow identification of disease markers for early diagnosis and treatment of progressive MS.

MS pathogenesis is driven by activation, expansion and infiltration of leukocytes into the CNS tissue. Accumulation of these leukocytes in perivascular cuffs at the blood-CNS interface, and their cellular interactions with resident glia within the CNS orchestrate a neuroinflammatory response that leads to immune-mediated demyelination [6, 7]. Intriguingly, while innate and adaptive immune cells promote a pro-inflammatory milieu causing neurodegeneration in MS, they also play a pivotal role in the resolution of immune-mediated attack and facilitate tissue repair [8-10]. This diverse role of activated leukocytes and microglia reflects their heterogeneity across a spectrum of pro-inflammatory to reparative phenotype. Accumulating evidence suggests that the net inflammatory balance of immune response is largely determined by the microenvironment [10]. Thus, it is critical to unravel endogenous mechanisms that regulate the phenotype of immune response during the onset, progression and reparative stages of MS. Identifying regulatory mechanisms implicated in early stage of MS pathogenesis would aid in early diagnosis, disease prevention and personalized therapeutic approaches.

Neuregulin-1 (Nrg-1) is a signaling protein that plays important roles in development and physiology of the peripheral and central nervous systems [11]. Nrg-1 is conventionally known for its critical role in oligodendrocyte development and myelination. However, in recent years, Nrg-1 has emerged as a new immune modulator in traumatic and ischemic CNS injuries [12-18]. Nrg-1beta1 (Nrg-1β1) is a major Nrg-1 isoform in the CNS that contains the epidermal growth factor (EGF) like domain; the functional domain of all Nrg-1 isoforms [19, 20]. In traumatic spinal cord injury (SCI) and lysolecithin-induced focal demyelinating lesions of the spinal cord, we have recently shown that Nrg-1β1 is dysregulated in these lesions, and its availability promotes oligodendrogenesis and remyelination [12, 21]. Moreover, we and others have shown that Nrg-1β1 attenuates astrocyte reactivity and the pro-inflammatory response of microglia in CNS injuries by blocking TLR/Myd88/NF-κB axis [13, 14, 22].

Efforts have been also made to identify the importance of Nrg-1 in MS. Earlier studies showed loss of Nrg-1 expression in MS active lesions [23], decreased expression in lysolecithin induced focal demyelinating lesions [12], reduced expression of one of its binding receptor ErbB4 in human blood mononuclear cells in RRMS patient samples [24], and an association of promoter polymorphisms in Nrg-1 gene with SPMS and PPMS patients [25]. Moreover, administration of the Nrg-1 isoform glial growth factor 2 (GGF2) promoted remyelination in a chronic relapsing mouse model of EAE [26]. Taken together, although early work studied Nrg-1 in MS, there exists a significant gap in our knowledge about its expression profile within the CNS and peripherally during the course of the disease, and its impact on pathogenesis, progression and recovery in autoimmune mediated demyelination.

### SUMMARY OF THE INVENTION

According to an aspect of the invention, there is provided a method of treating or prophylactically treating multiple sclerosis comprising:
administering to an individual in need of such treatment an effective amount of Nrg-1β1.

According to another aspect of the invention, there is provided a method of treating multiple sclerosis comprising:
administering to an individual in need of such treatment an effective amount of Nrg-1β1.

According to another aspect of the invention, there is provided a method of prophylactically treating multiple sclerosis comprising:
administering to an individual in need of such treatment an effective amount of Nrg-1β1.

According to a further aspect of the invention, there is provided use of Nrg-1β1 for treatment or prophylactic treatment of multiple sclerosis.

According to yet another aspect of the invention, there is provided use of Nrg-1β1 for preparation of a medicament for treatment or prophylactic treatment of multiple sclerosis.

According to another aspect of the invention, there is provided Nrg-1β1 for treatment or prophylactic treatment of multiple sclerosis.

According to a further aspect of the invention, there is provided Nrg-1β1 for preparation of a medicament for treatment or prophylactic treatment of multiple sclerosis.

According to another aspect of the invention, there is provided a method for determining if an individual is a candidate for prophylactic treatment for multiple sclerosis with Nrg-1β1 comprising:
measuring an Nrg-1β1 level in a sample from the individual,
wherein, if the Nrg-1β1 level is below a threshold value, the individual is a candidate for Nrg-1β1 treatment.

According to another aspect of the invention, there is provided a method for determining if an individual is a candidate for further assessment for multiple sclerosis comprising:
measuring an Nrg-1β1 level in a sample from the individual,
wherein, if the Nrg-1β1 level is below a threshold value, the individual is further assessed for multiple sclerosis.

According to another aspect of the invention, there is provided a method for determining if treatment of an individual for multiple sclerosis is successful comprising:
taking a first sample from the individual;
measuring a first Nrg-1β1 level in the first sample from the individual,
then administering a treatment for multiple sclerosis to the individual for a period of time;
after said period of time, taking a second sample from the individual and measuring a second Nrg-1β1 level in the second sample from the individual; and
comparing the first Nrg-1β1 level to the second Nrg-1β1 level,
wherein if the second Nrg-1β1 level is higher that the first Nrg-1β1 level, the treatment is successful.

### BRIEF DESCRIPTION OF THE DRAWINGS

FIG. 1: Nrg-1β1 expression levels are declined in CNS and peripherally in EAE mice. (A) Representative luxol fast blue and hematoxylin (LFB-HE) stained spinal cord tissue from naïve and EAE mice at the peak of the disease indicating demyelinating lesions. (B) Immunohistological examination for myelin (MBP) and Nrg-1β1 revealed that Nrg-1β1 expression is depleted in demyelinated regions whereas adjacent myelinated normal appearing white matter area (NAWM) as well as naive mice tissue indicated a strong expression of MBP and Nrg-1β1. (C) Quantitative immunofluorescence intensity in EAE spinal cord lesions showed 48% reduction in Nrg-1β1 as compared to the adjacent NAWM and naïve mice tissue. Values are represented as fold change in intensity normalized to naïve. (D-F) Longitudinal assessment of Nrg-1β1 levels was performed on spinal cord (D), plasma (E) and spleen (F) of EAE mice at 7 days post induction (7 dpi), onset (10 dpi), peak (14-16 dpi), 7 days post peak (dpp), 14 dpp and 28 dpp. Nrg-1β1 was significantly depleted in plasma, spleen and spinal cord of EAE mice at 7 dpi, onset and peak of the disease. It was restored in plasma and spleen during the recovery phase (7 dpp, 14 dpp and 28 dpp). However, in the spinal cord there was another reduction Nrg-1β1 levels at 14 dpp in which persisted until 28 dpp. Values represent mean±standard error mean (SEM). *p<0.05; One-way ANOVA followed by Holm-Sidak post-hoc test. N=3-5. Naïve mean values were compared to each time point for post-hoc test in D, E and F.

FIG. 2: Nrg-1β1 treatment ameliorates neurological disability in EAE mice. (A) Mice were assessed daily for EAE symptoms on the basis of tail and hind limb functional deficits. Treatment with recombinant human Nrg-1β1 peptide (300 ng, 600 ng and 1200 ng/day) was administered subcutaneously (s.c.) starting at the peak of the disease (day 16 post induction) for 4 weeks. Nrg-1β1 treatment improved functional deficits in a dose dependent manner in EAE mice. Daily clinical scores were expressed as mean±SEM, *p<0.05. Two-way-ANOVA followed by Holm-Sidak post-hoc test. (B) Cumulative disease burden for each animal was calculated as area under the curve. 600 ng and 1200 ng/day Nrg-1β1 treated groups showed significant reduction in their mean cumulative disease burden as compared to vehicle treated group. *p<0.05. N=10 for Vehicle, 300 ng and 600 ng/day Nrg-1β1 groups. N=5 for 1200 ng/day Nrg-1β1 group. (C) Clinical score of each mouse in vehicle and Nrg-1 treatment group (600 ng) was plotted as heat map. Sustained daily Nrg-1β1 treatment (600 ng/day) significantly improved clinical score and reduced the cumulative burden of disease when administered at different paradigms including symptomatically at the onset of EAE (D-F), prophylactically at the time of EAE induction (G-I), and delayed at 4 days after reaching the peak of the disease (J-L). (M-O) Transient Nrg-1β1 therapy for 7 days starting at peak of EAE did not confer beneficial effects when assessed. Clinical scores are expressed as average (mean±SEM). *p<0.05. Two-way-ANOVA followed by Holm-Sidak post-hoc test. N=7-10. *p<0.05; Mann-Whitney non-parametric test for area under curve graph statistics.

FIG. 3: Nrg-1β1 treatment attenuates leukocyte infiltration and inflammation foci in the spinal cord of EAE mice. (A) Representative images of Luxol Fast Blue and hematoxylin/eosin (LFB-HE) stained spinal cord tissue show active inflammatory and demyelinating lesions (black arrows) in the white matter (WM) from vehicle and Nrg-1β1 treated animals 2 weeks after peak treatment. (B-C) Nrg-1β1 treatment significantly reduced the area and number of EAE lesions as compared to the vehicle group. *p<0.05; Student's t-test. N=5. (D) Representative images of perivascular and spinal cord tissue stained with leukocyte marker-CD45 and laminin in naïve, vehicle and Nrg-1 treated group. (E) Higher magnified images of CD45+/DAPI+ in the spinal cord. (F) Infiltrating CD45+ cells were significantly reduced after Nrg-1β1 treatment as compared to vehicle group. *p<0.05; Student's t-test. N=5. (G-I) Multiplex Mesoscale ELISA at 2,7 and 14 dpp revealed that Nrg-1β1 treatment significantly reduces the expression levels of chemokines involved in (G) recruitment of neutrophils-CXCL1/2 (Keratinocyte chemoattractant KC/human growth-regulated oncogene KC-GRO), (H) monocyte chemotactic protein-1 (MCP-1) and (I) chemokine for T cells—CXCL10 (Interferon-γ-Inducing Protein-10). Data represent mean±SEM, *p<0.05; Student's t-test. N=4-8. (J) Gelatin zymography for matrix metalloprotease (MMP)-2 and -9 activity was performed on the spinal cord lysate samples of EAE mice treated with vehicle or Nrg-1β1. MMP-9 activity was significantly elevated as the result of EAE, which was significantly reduced by Nrg-1β1 treatment to a level close to the basal level detected in naïve non-EAE spinal cord. (K) No alteration in activity of MMP-2 was observed in vehicle or Nrg-1β1 treated group as compared to the naïve group. (L) Representative gelatin zymogram of MMP-2 and -9 activity in the spinal cord lysate samples of EAE mice treated with vehicle or Nrg-1β1. Data represents mean fold change in expression ±SEM normalized to the naïve group. *p<0.05. One-way ANOVA followed by Holm-Sidak post-hoc test. N=4-6.

FIG. 4: Nrg-1β1 treatment attenuates the expression of chondroitin sulphate proteoglycans (CSPGs) in EAE lesions. (A) Representative images of immunohistochemical staining of CSPGs, microglia/macrophage (Iba-1) and astrocytes (glial fibrillary acidic protein, GFAP) in naïve, vehicle and Nrg-1β1 treated groups. Treatments were administered for 2 weeks starting at peak of the disease. (B) CSPGs were highly upregulated in association with both astrocytes and microglia/macrophages as demonstrated by co-labelling with Iba-1 and GFAP in the high-magnification images of marked area. Yellow arrows show co-labelling with Iba-1 and GFAP respectively. (C-D) Quantitative immunofluorescence intensity and slot blot analysis showed Nrg-1β1 treatment significantly abated the EAE-induced expression of CSPGs. Data represents mean fold change in expression ±SEM normalized to the naïve group. *p<0.05. One-way ANOVA followed by Holm-Sidak post-hoc test. N=4-5.

FIG. 5: Nrg-1β1 suppresses monocyte expansion and infiltration and attenuates pro-inflammatory phenotype of microglia and macrophages in EAE mice. (A) Flow cytometric analysis of spinal cord from vehicle and Nrg-1β1 treated animals at 7 dpp showed that Nrg-1β1 did not change the overall population of CD3−/CD11b+ microglia and macrophages. (B-C) Immunohistochemical cell density analysis of microglia/macrophage common marker, Iba-1 and microglia specific marker TMEM119 also confirmed that Nrg-1β1 did not alter the recruitment/activation of macrophages and resident microglia in the spinal cord as compared to the vehicle EAE groups. *p<0.05. One-way ANOVA, N=3-4. (D) Representative images of TMEM119 immunostaining from spinal cord lesions of naïve, vehicle and Nrg-1 treated groups are shown. (E-F) Nrg-1β1 treatment significantly reduced circulating monocytes (CD11clo/CD11bhi/Ly6g−/NK1.1−) in the blood and infiltrating macrophages (CD3−/CD49e+/CD11c−/Ly6c+) in the spinal cord of Nrg-1β1 treated animals as compared to vehicle group. (G-H) Nrg-1β1 treatment also significantly reduced pro-inflammatory “M1” (CD3-/CD11b+/CD80+) microglia/macrophages, while promoted an anti-inflammatory “M2” (CD3−/CD11b+/CD206+) phenotype. (I-J) Monocyte derived “M1” macrophages (CD3−/CD49e+/CD80+) were also decreased in the spinal cord Nrg-1β1 treated mice, while “M2” macrophages were increased (CD3−/CD49e+/CD206+). (K-L) Representative images of EAE spinal cord immunostained with “M1” marker (CD80) or “M2” marker (CD206) co-labelled with microglia/macrophage markers Iba-1 or OX-42, respectively, show the “M1” to “M2” phenotype shift in microglia/macrophage population in Nrg-1β1 treated group as compared to vehicle group at 7 dpp time-point. (M) Flow cytometric assessment showed a significant reduction in total antigen presenting cells (APCs, CD3-IA/IE+) in the EAE spinal cord under Nrg-1β1 treatment as compared to vehicle group. (N-P) Cytokine analysis by ELISA in spinal cord tissues also revealed Nrg-1β1 treatment significantly reduced pro-inflammatory cytokines Interleukin (IL)-113, tumor necrosis factor alpha (TNF-α), and IL-6. (Q-R) Reactive oxygen species (ROS) was detected in EAE mice by conversion of dihydroethidium (DHE) to ethidium in the spinal cord tissue. EAE resulted in substantial increase in ROS levels in the white matter of the spinal cord in which was significantly reduced by Nrg-1β1 treatment. (S) Slot blot analysis of oxidized lipids (E06) was performed on spinal cord lysates at 14 days after Nrg-1β1 treatment. EAE induced E06 levels, which was reduced significantly after Nrg-1β1 treatment as compared to the vehicle treated group. Data represents mean±standard error mean (SEM). *p<0.05; Student's t-test. N=6-8.

FIG. 6: Nrg-1β1 treatment promotes a T regulatory response directly and indirectly by modulating macrophages. (A-B) Flow-cytometry of vehicle and Nrg-1β1 treated mice after 7 days of treatment revealed no change in the total number of CD4+ T cells in the blood and spinal cord. (C-D) While the total number of effector T cells (CD4+IFNγ+) remained unchanged in the blood, there was a significant reduction in CD4+IFNγ+ cells in the spinal cord of Nrg-1β1 treated animals as compared to vehicle group. (E-G) Cytokine assessment in spinal cord tissue with ELISA showed that Nrg-1β1 treatment significantly reduced key drivers of Th1 cell differentiation interferon gamma (IFNγ) and interleukin (IL)-2 and IL-16. *p<0.05; Student's t-test. N=6-8. (H-I) Flow-cytometry for CD4+IL-17+ effector T cell population after 7 days of Nrg-1β1 treatment did not show any change in this population in the blood or spinal cord. (J-K) A significant increase in anti-inflammatory T regulatory (CD4+/CD25+/FR4+) and CD4+/CD25+/FoxP3+ cells was observed with Nrg-1β1 treatment as compared to vehicle group. *p<0.05; Student's t-test. N=6-8. (L-O) Naïve CD4+ T cells were polarized in vitro under Th1 conditions and were cultured with different concentration of Nrg-1β1, conditioned medium (CM) from normal (M0) and M1 polarized (IFNγ+LPS treated) microglia and bone marrow derived macrophages (BMDM) treated with Nrg-1β1 50 ng/ml or 200 ng/ml for 72 hrs. (L) Flow cytometric assessment revealed higher dose of Nrg-1β1 (200 ng/ml) significantly reduced CD4+IFNγ+ T cells. (M) While there was a significant increase in CD4+IFNγ+ cell population under BMDM M1 CM, Nrg-1β1 200 ng/ml treated M1 BMDM CM was able to diminish this increase significantly. (N) M0 Microglial CM reduced the total number of Th1 polarized CD4+IFNγ+ T cells, (0) while astrocytes CM did not alter Th1 cell population. *p<0.05. One-way ANOVA followed by Holm-Sidak post-hoc test. N=4-5.

FIG. 7: Proteomic analysis asserts that lipid oxidation and immune modulation are key Nrg-1β1 mediated mechanisms in EAE recovery. (A) Volcano plot illustrates differentially abundant proteins in the spinal cord of vehicle and Nrg-1β1 treated mice at 7 days post-peak of EAE. The −log 10 is plotted against the log 2 (fold change). The horizontal line denotes P=0.05, which was set as significance threshold (prior to logarithmic transformation). (B) Functional analysis of differentially expressed proteins using ClueGo plug-in in cytoscape software shows the interactions among the significantly different biological functions associated with upregulated and downregulated proteins in this study. Based on the κ score level, biological functions are depicted as colored nodes linked to related groups. (C) Differentially expressed proteins were further analyzed using DAVID software. Left axis represents the fold enrichment of each biological function GO term. Only statistically significant GO terms are shown. Key for the GO terms are: GO:0098609-cell-cell adhesion; GO:0098641-cadherin binding involved in cell-cell adhesion; GO:0005913-cell-cell adherens junction; GO:0034440-lipid oxidation; GO:0005777-peroxisome; GO:0019395-fatty acid oxidation; GO:0009062-fatty acid catabolic process; GO:0006635-fatty acid beta-oxidation; GO:0004721-phosphoprotein phosphatase activity; GO:0006470-protein dephosphorylation; GO:0043123-positive regulation of I-kappaB kinase/NF-kappaB signaling; GO:0045087-innate immune response; GO:0033555-multicellular organismal response to stress; GO:0019771-negative regulation of cell morphogenesis involved in differentiation.

FIG. 8: Nrg-1β1 is depleted in plasma and brain lesions of MS patients. (A) Post-mortem brain samples from MS patients were stained for luxol fast blue and hematoxylin/eosin (LFB-HE) to identify demyelinating lesion in the white matter (B) Representative images of normal appearing white matter (NAWM) or lesion area from MS brain sections stained with antibodies against Nrg-1β1 and myelin basic protein (MBP). (C) Quantification for Nrg-1β1 immunofluorescence intensity was performed from 6 different MS brain samples comparing the Nrg-1β1 intensity in lesion to the NAWM from same section. Values are represented as fold change in intensity normalized to NAWM for each sample which is shown as dotted baseline. There was 39% reduction in Nrg-1β1 expression within MS lesions as compared to the adjacent NAWM. (D-G) ELISA was performed for Nrg-1β1 on human plasma samples from normal individuals and MS patients. (D) Box plots show range of Nrg-1β1 levels (minimum to maximum) in normal controls (HC, N=30) and MS patients (N=136). (E) MS patients were categorized into disease modifying therapy (DMT, N=88) or no DMT (N=48) users at the time of sample collection. Note: Nrg-1β1 levels of DMT users are depicted as box plots filled with pattern. (F) Clinically isolated syndrome (CIS) individuals showed a significant reduction in Nrg-1β1 levels in plasma as compared to normal individual samples. *p<0.05; Mann-Whitney U-test. (G) Nrg-1β1 plasma levels of CIS individuals were further categorized based on their subsequent diagnosis of MS (RRMS) in the follow-up years and compared to normal patient samples. 6 of 11 CIS patients converted to RRMS and represented lower levels of Nrg-1β1 as compared to those who did not progress to MS (non-converter). (H) MS patients were further categorised on the basis of clinical diagnosis into different MS type/stage and whether they received any DMT. Nrg-1β1 levels in plasma samples from CIS (N=11; NDMT=7, DMT=4)], RR MS (N=113; NDMT=31, DMT=82] and secondary progressive MS [SPMS (N=12; NDMT=10, DMT=2)] were analyzed. Nrg-1β1 levels in CIS and SPMS patients who did not receive DMT were significantly reduced as compared to normal individuals. *p<0.05; Mann-Whitney U-test. (I) Nrg-1β1 plasma levels of normal individuals and MS patients (with or without DMT) was analyzed against their expanded disability status scale (EDSS). Nrg-1β1 levels were stable in DMT receiving patients irrespective of EDSS score, while MS patients without any DMT demonstrated lower levels of Nrg-1β1 as compared to normal individuals. ‘+’ depicts the mean value of Nrg-1β1 levels among analyzed samples.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As discussed herein, using MS patient samples and the MS mouse model of experimental autoimmune encephalomyelitis (EAE), we report that Nrg-1β1 expression was dysregulated early in the course of the disease. In a cohort of MS patients with various sub-types of the disease, we demonstrate for the first time that plasma levels of Nrg-1β1 were significantly reduced in CIS individuals and its reduction was associated with an increased likelihood (55%) of subsequently being diagnosed with RRMS. Similarly, in the EAE mice, Nrg-1β1 was significantly reduced in the plasma at pre-symptomatic phase and this persisted during EAE onset and progression. Nrg-1β1 expression was also severely depleted in active demyelinating lesions of MS and EAE. Importantly, dysregulation of Nrg-1β1 appeared to functionally impact EAE progression, as restoring its deficient levels in EAE mice through systemic administration delayed EAE symptoms and ameliorated severity of EAE symptoms. Nrg-1β1 exhibited an extended therapeutic time window, as it was effective when administrated prophylactically, symptomatically, acutely or chronically. Mechanistically, availability of Nrg-1β1 promoted a comprehensive immune regulatory response by myeloid cells and T helper type 1 (Th1) cells in EAE. Our flow cytometry, cytokine profiling and proteomics showed that Nrg-1β1 moderated monocyte infiltration and several key mediators of EAE immunopathogenesis and neurodegeneration. Taken together, our findings establish a novel supportive role for Nrg-1β1 in MS pathogenesis and demonstrate its use as an early disease biomarker and a therapeutic target in MS. Identifying regulatory mechanisms implicated in early stage of MS pathogenesis would aid in early diagnosis, disease prevention and personalized therapeutic approaches.

Currently, little is known about the early endogenous mechanisms that regulate MS autoimmune response at the onset and progression of the disease. Understanding MS mechanisms can aid in identifying disease markers and facilitate clinical assessment, timely diagnosis and treatment of MS. In the present study, utilizing the preclinical EAE mouse model and MS patient samples, we provide new evidence that dysregulation of Nrg-1β1 is associated with MS pathology and is detectable both peripherally in the plasma and centrally in the CNS lesions. Downregulation of Nrg-1β1 precedes EAE symptoms and persists during disease onset and progression, when the immune response is predominantly pro-inflammatory. Importantly, we provide evidence that downregulation of Nrg-1β1 has impact on EAE immunopathogenesis, as therapeutic restoration of Nrg-1β1 in the EAE mice delayed disease onset and attenuated clinical severity of EAE. Relevance of these findings to MS was corroborated by detecting lower levels of Nrg-1β1 in the plasma of CIS individuals at the onset of MS compared to normal individuals. Capitalizing on these new findings, we propose that downregulation of Nrg-1β1 is a disease characteristic in early MS and its reduced levels may indicate disease severity. This notion is furthered supported by our findings that MS patients whose disease was regulated with DMTs had a more normal level of Nrg-1β1 in their plasma. While efforts are being made to identify early disease markers for MS or to develop treatments that can prevent or delay progression to definite MS [47], Nrg-1β1 appears to be a promising target for further investigations in this direction.

Nrg-1 is well-known for its diverse roles in the development and physiology of the nervous system [11]. Nrg-1β1 isoform is predominantly expressed in the nervous system [48]. In the CNS, Nrg-1 is highly expressed by neurons and is axonally-localized [21, 49]. Likewise, oligodendrocytes express Nrg-1 in the injured and healthy rodent spinal cord tissue [21, 50]. Nrg-1 is also expressed by astrocytes to a lesser extent, where intracellular cAMP levels and protein kinase C (PKC) signaling pathways have been shown to regulate its expression in vitro [29]. Expression of Nrg-1 mRNA has been also reported in peripheral blood mononuclear cells (PBMCs) and in mouse brain [29, 51], although our previous studies did not show a detectable level of Nrg-1 protein in microglia/macrophage in the spinal cord (Gauthier et al., 2013). All known secreted isoforms of Nrg-1 contain a heparin-binding domain that binds to heparan sulfate proteoglycan (HSPG) and acts as a highly specific targeting mechanism to deliver Nrg-1 to the extracellular matrix (ECM) of sites where it is needed, such as developing white matter tracts of the spinal cord and the basal lamina of neuromuscular synapses [52, 53]. Interestingly, Nrg-1 precursors are produced in cortical neurons, while soluble Nrg-1 ligand becomes concentrated within the extracellular matrix of white matter, where it can be released into the cerebrospinal fluid (CSF) [54]. Although the underlying cause of Nrg-1β1 downregulation in EAE lesions needs further elucidation, our data suggest its reduction in EAE lesion may reflect degeneration of its primary sources, axons and oligodendrocytes. This observation is reminiscent of our previous studies in traumatic SCI and lysolecithin (LPC)-induced focal demyelinating lesions that also showed a long lasting reduction in Nrg-1β1 protein expression in white matter lesions of the spinal cord [12, 21]. An early study also reported absence of Nrg-1 in active MS lesions, which was attributed to astrocytes, although the conclusion was made qualitatively [23]. Importantly, we have shown for the first time that Nrg-1β1 was more robustly but transiently downregulated peripherally in the spleen and blood of EAE mice during the pre-symptomatic, onset and peak of the disease, as compared to its persistent but less severe dysregulation in the spinal cord. Further investigations are needed to determine how Nrg-1β1 is transiently depleted in the spleen and blood circulation in early and acute stages of EAE. However, our data support the plausibility of an active suppression of Nrg-1β1 expression rather than extravasation of Nrg-1β1 expressing leukocytes to the CNS, as the decline was simultaneously observed in the EAE lesions. Nonetheless, transient dysregulation of Nrg-1β1 in the spleen and blood during EAE pathogenesis points to its importance as an early disease characteristic and a potential immunotherapeutic target.

Therapeutically, we demonstrate that subcutaneous rh Nrg-1β1 treatment delayed EAE onset and alleviated disease progression and severity, when administered prophylactically, symptomatically, acutely or chronically. An early study by Canella and colleagues in 1998 also examined the therapeutic effects of administering glial growth factor-2 (GGF2, a 40 kDa isoform of Nrg-1), in a chronic relapsing SJL/J mouse model of EAE [26]. These studies showed that subcutaneous administration of 2 mg/kg dose of rhGGF2 acutely at the time of EAE induction delayed EAE symptoms and significantly reduced relapses. Treatment at the peak of disease also reduced relapses; however, it had no apparent effects on mean clinical scores in the EAE mice. Of note, effective dose of rh Nrg-1β1 for EAE neurological recovery in our study was 600 ng/day/mouse or 30 μg/kg which is much lower compared to the range of 0.2 to 2 mg/kg dose of rhGGF2 in these studies [26]. Beneficial effects of rhGGF2 on clinical recovery was accompanied by improved remyelination in EAE mice. This work, however, did not study either the expression profile of GGF2 during the course of EAE or its role in pathogenesis of EAE. Thus, it would be intriguing to know whether GGF2 follows the same expression profile and pathological characteristics centrally and peripherally in EAE and MS as what we have identified for Nrg-1β1 in our studies

We have uncovered that the beneficial therapeutic effect of Nrg-1β1 is associated with several immune regulatory mechanisms in EAE. Regulation of monocyte response appeared to be a major mechanism, as Nrg-1β1 treatment specifically suppressed circulating monocytes and reduced their infiltration into EAE lesions, while having had no apparent effects on the number of circulating or infiltrated T cells. These results were well supported by our chemokine profiling in which key monocyte chemoattractants, CXCL1/2, CXCL10 and MCP-1, were significantly reduced in Nrg-1β1 treated EAE mice. It is well-established that pro-inflammatory monocyte-derived macrophages accumulate in EAE lesions during onset and peak of the disease [36, 37] and drive autoimmune mediated demyelination by producing cytokines and presenting myelin epitopes to activating CD4+ T cells [32, 55-57]. Reducing monocyte infiltration and activation has previously improved clinical scores in EAE mice [58]. Moreover, blocking monocyte entry into the CNS in Ccr2 null mice delayed EAE onset, while enhancing monocyte infiltration via SOCS3 deficiency accelerated disease onset and exacerbated neurological disability in EAE [59, 60].

Mechanistically, our findings suggest that Nrg-1β1 may suppress monocyte infiltration by its remarkable ability to reduce the activity of CSPGs and MMP-9. Recent studies in MS and EAE uncovered that CSPGs facilitate leukocyte accumulation in the perivascular cuff and promote their trafficking into the CNS [32, 33]. Our studies in SCI also identified a pro-inflammatory role for CSPG signaling [61]. Leukocyte infiltration also requires activation of MMPs that are expressed by microglia, monocytes and macrophages [8, 31, 62]. MMP-9, in particular, plays a key role in disruption of the blood-CNS barrier and promoting MS pathogenesis [63, 64] [65]. As supporting evidence, studies in cortical injury showed that Nrg-1 treatment reduces injury-induced permeability of endothelial cells in the blood-CNS barrier by attenuating IL-1β [66]. Of interest, our previous studies in SCI and LPC-induced focal lesions also showed a reduction in CSPGs production and MMP-9 activity in the spinal cord under Nrg-1β1 treatment [12, 13], suggesting a common immunomodulatory mechanism for Nrg-1β1 in CNS inflammation. CSPGs and MMP-9 can be produced by multiple cell types in EAE and other inflammatory conditions including activated astrocytes, microglia and infiltrating monocyte derived macrophages [32, 67-69]. Moreover, as shown in our study and previous reports, these cells express Nrg-1β1 binding receptor ErbB2 and ErbB4 under normal and inflammatory conditions. Thus, Nrg-1β1 could potentially attenuates the production of MMP-9 or CSPGs directly by influencing activated astrocytes, microglia and macrophages in EAE. However, further studies with cell specific targeted approaches are warranted to dissect the role of Nrg-1β1 in regulating the expression of CSPGs and MMP-9 under inflammatory microenvironment.

Interestingly, Nrg-1β1 did not influence microglia recruitment into EAE lesions, while it fostered a phenotype shift in CD11b+microglia and macrophages towards anti-inflammatory “M2”-like phenotype with a concomitant decrease in pro-inflammatory “M1”-like cells. This is a desirable therapeutic outcome in EAE and MS, as microglia and macrophages are also critical in facilitating remission in MS [44]. Heterogeneity of microglia and their diverse activated phenotype is increasingly recognized in MS pathophysiology [70]. Recent work showed that microglia even attenuate the toxic effects of macrophages in demyelinating lesions [70]. Depletion of “M2”-like microglia and macrophages has impaired remyelination [44, 45], and our previous in vitro studies also identified that availability of Nrg-1β1 can restore the suppressed phagocytic properties of pro-inflammatory microglia [15], which is a prerequisite for successful repair and remyelination. Collectively, our findings support a positive role for Nrg-1β1 in fostering a reparative phenotype in microglia. The positive effects of Nrg-1β1 on microglia phenotype may explain the results of previous studies by our group and others in rodent models of CNS injury and demyelination that showed Nrg-1 promotes endogenous oligodendrogenesis, preserves axons and promotes spontaneous remyelination [12, 13, 21, 26]. However, further studies are needed to elucidate the specific effects of Nrg-1β1 on neurons, axons, and oligodendrocytes in the context of EAE.

We demonstrate that Nrg-1β1 specifically regulated IFN-γ+Th1 effector cells in EAE mice without any apparent role in Th17 response. Interestingly, unlike monocytes, Nrg-1β1 regulation of Th1 Nrg-1β1 influenced Th1 polarization directly and indirectly through modulation of macrophages. These findings are supported by our previous study, in which systemic Nrg-1β1 suppressed IFN-γ+ effector T cells in traumatic SCI in rats [14]. IFN-γ is implicated in the pathogenesis of MS and EAE and its intrathecal administration promotes early disease onset in EAE [71]. The effects of IFN-γ on APCs are pleiotropic and encompass up-regulation of MHC molecules, induction of ROS, phagocytic activity, and increased production of pro-inflammatory cytokines. In fact, EAE is dependent on IFNγ induced production of MCP1 (CCL2) and CXCL10 that facilitate monocytes infiltration into the white matter [72]. Thus, reduction of IFN-γ+Th1 effector cell population and downregulation of MCP1 and CXCL10 chemokines appear to be an underlying mechanism by which Nrg-1β1 regulated EAE progression and recovery in our studies.

Nrg-1β1 promoted a Treg response in EAE. Treg cells are known to suppress proliferation and activation of Teff cells by inhibiting autoreactive T cells [73, 74]. Importantly, Treg cells mediate recovery from EAE by attenuating the cytokine production, proliferation and motility of effector T cells in the CNS [75]. These reports support our findings in this study, where Nrg-1β1-induced increase in Treg population was accompanied by reduced Th1 cells and diminished pro-inflammatory cytokine production in the EAE mice. We previously observed that Nrg-1β1 promotes upregulation of regulatory cytokine IL-10 in SCI and LPC focal demyelination [12, 14]. However, intriguingly, we did not detect any changes in IL-10 expression in this study indicating an IL-10 independent immunomodulatory mechanism of Nrg-1β1 in our EAE model. Interestingly, a missense mutation in Nrg-1 gene has been associated with immune dysregulation in schizophrenia [76]. Individuals carrying the mutation showed significantly elevated levels of IL-113, IL-6, IL-10, and TNF-α in plasma [76], demonstrating a direct association between dysregulation of Nrg-1 and immune cell over-activation and cytokine production. Taken together, based on our new findings in EAE, we propose that endogenous Nrg-1β1 is important for immune homeostasis and its dysregulation is a disease mechanism that facilitates EAE onset and progression by promoting monocyte extravasation and inducing an IFN-γ Th1 response. To address this hypothesis, future conditional knockout studies are required to elucidate whether the absence of Nrg-1β1 would result in immune dysregulation peripherally and in the CNS.

A major disadvantage of available immunosuppressive therapies in MS is that they generally impair T-cell functions that can adversely increase the risk for systemic infections and comorbidities [77]. Identifying specific immune regulatory mechanisms of MS pathology would allow development of targeted treatments. Our work has identified an endogenous pathway that appears to play a role in immune homeostasis, and that its disruption is associated with MS pathogenesis. In addition to its potential as an early disease marker, Nrg-1β1 represents a desirable specific immune regulatory therapy, as its restoration can moderate the imbalanced immune response and disease severity in EAE.

We have identified several potential therapeutic advantages of Nrg-1β1 treatment for MS.

Firstly, this treatment is aimed to restore the dysregulated levels of endogenous Nrg-1β1 and not over-activating a pathway that may result in adverse effects.

Secondly, Nrg-1β1 regulates the phenotype of innate and adaptive immune cells rather than suppressing the immune response.

Thirdly and intriguingly, Nrg-1β1 treatment offers an extended therapeutic time window at least in EAE mice by showing efficacy when administered at various points during the course of the disease.

Lastly, an important property of Nrg-1β1 peptide is its desirable pharmacokinetics for CNS therapeutics, as its ability to pass the blood-CNS barrier is confirmed [30].

As will be appreciated by one of skill in the art, research in multiple sclerosis utilizes several animal models to investigate MS pathogenesis, disease mechanisms and therapeutics. As discussed herein, while these models share the demyelinating aspect of MS pathology, they differ from each other and provide an opportunity to study various mechanisms of MS in a complementary fashion. Among these models, mouse experimental autoimmune encephalomyelitis (EAE) is currently the closest animal model to MS due to its auto-immune mediated myelin pathology and is considered the most clinically relevant animal model for therapeutic development for MS. In our MS research, we have employed the mouse EAE model along with a rat model of lysophosphatidylcholine-induced demyelination.

Lysophosphatidylcholine or lysolecithin induced demyelination model is a toxin mediated focal demyelination model. Intraparenchymal focal injection of LPC toxin into the brain or spinal cord induces cell death in myelinating oligodendrocytes resulting in loss of myelin (demyelination). LPC targets myelin primarily because it has a specific affinity for lipids in the cell membrane and myelin is known as a membranous structure with a high lipid content. Shortly after injection (within 3 days), LPC causes myelin lamellae to fuse, transform into spherical vesicles and progressively reduce in size until they are eventually phagocytosed. While LPC causes demyelination, it lacks absolute cellular specificity to oligodendrocytes and myelin, as a reduction in astrocytes and axons is also observed to a lesser extent in LPC induced focal lesion. We have used LPC injection to induce focal demyelination in specific regions of the spinal cord white matter in rats for assessing the effect of various in promoting re-myelination treatments including Nrg-1β1. LPC also triggers infiltration of macrophages/microglia, activation of astrocytes and oligodendrocyte precursor cells and axonal injury within the focal demyelinating lesions. These cellular changes are followed by spontaneous remyelination that is complete by 3-4 weeks post LPC injection. Thus, this model has been used extensively to interrogate the complex mechanisms of demyelination and remyelination.

The disadvantage of the LPC model is that oligodendrocyte cell death and demyelination is not induced by an immune response and, accordingly, it is not physiologically relevant and does not reflect MS pathogenesis, disease progression or clinical phenotype. Importantly, LPC mediated demyelination does not result in any functional impairment or neurological symptoms, as the lesion is small and restricted to the injection site in the brain or spinal cord tissue. Due to the lack of auto-immune response and functional deficits, the LPC model is not suitable for therapeutic development for MS and is primarily utilized to study the mechanisms of remyelination.

In contrast, experimental autoimmune encephalomyelitis (EAE) is the most commonly used animal model in MS research and has contributed towards the development of a number of first-line immunomodulatory treatments for MS patients. EAE is an auto-immune disease of the central nervous system (CNS), following the induction of the immune response against CNS-specific antigens. In MS, EAE is commonly induced by using myelin peptides such as proteolipid protein (PLP), myelin basic protein (MBP) and myelin oligodendrocyte glycoprotein (MOG). These peptides are usually emulsified in incomplete Freund's adjuvant (IFA) alongside mycobacterium to make complete Freund's adjuvant (CFA). The use of adjuvants aims to mimic the immune activation pathways caused by infectious agents and increase the efficiency of EAE induction Immunization results in the activation of myelin antigen-specific T cells in peripheral immune organs outside of the CNS and their subsequent proliferation and differentiation into effector T cells. The expression of integrins on these effector T cells enables them to cross the blood-CNS barrier. Once activated T cells enters the CNS tissue, they are re-activated by resident myelin antigen-presenting cells (APCs), which results in expression of pro-inflammatory cytokines by effector T cells. Furthermore, production of pathogenic chemokines recruits immune cells into the CNS. These immune auto-mediated processes are largely responsible for the destruction of myelin sheath (demyelination), which presents as ascending paralysis, starting at the tail, followed by the hind limbs, and progressing onto the upper limbs. In mouse EAE model described herein, animals show neurological symptoms around day 12 and reach the peak of the disease around day 16.

Disease onset is accompanied by infiltration of immune cells into the cortex, spinal cord and cerebellum. While pro-inflammatory auto-immune response is resolved to some extent and reduces neurological disability, the presence of immune cells persists up to 7-8 weeks post EAE induction. Moreover, neurological deficits associated with EAE are not resolved and animals show reduced mobility at the chronic stage that is associated with permanent axonal loss in the spinal cord lesions of EAE.

Summary of major differences between LPC and EAE:
In contrast to EAE, LPC induced demyelination results are due to lipid toxicity and not autoimmune mediated pathology
In contrast to EAE pathology that start peripherally (outside the CNS), LPC induced demyelination is not systemic and is generated by focal injections of the toxin into the CNS tissue resulting in focal isolated lesions. Specifically, LPC is a toxin that selectively and directly kills oligodendrocyte resulting in demyelination without any involvement of immune cells, while MS is an immune mediated disease in which immune system attacks the CNS myelin leading to demyelination (similar to what happens in EAE).
   In contrast to EAE mice who show MS like symptoms (i.e. neurological deficits), animals with LPC induced demyelination does not show any functional impairment. Therefore, LPC model is not suitable for assessing the potential of a treatment in improving neurological deficits associated with MS.
LPC induced focal demyelination is completely resolved in 3-4 weeks.
In contrast to LPC toxin that can induce demyelination in any rodent strain, EAE can be induced only in some susceptible rodent strains. Specifically, LPC injections produce only focal demyelination without any neurological deficits, while EAE can be modelled based upon type of encephalitogenic peptide, type of mouse strain and method of inductions, which mimic different types and phases of MS for preclinical studies.
One of the major advantages of the autoimmune mediated EAE model as compared to toxin (LPC) induced demyelination is that both pathological and repair processes occur simultaneously, which closely mimics the MS pathology and help in developing potential therapeutic strategies.

As will be appreciated by one of skill in the art, with EAE model we can study different phases (pre-onset, onset, peak of the disease, chronic) and types of MS (RRMS, SPMS). LPC addresses only remyelination aspect which is one of the events in MS. Additionally, the LPC model can not reveal an alteration/involvement in the peripheral immune response which is central to MS and can be only studies by EAE model.

In some embodiments, Nrg-1β1 peptide is a better alternative for treatment than a stabilized Nrg-1β1 peptide, which may elicit a prolonged activation of its signalling pathways with unknown long-term effects as Nrg-1β1 peptide has been studied in different rodent models without any known adverse effects.

According to an aspect of the invention, there is provided a method of treating or prophylactically treating multiple sclerosis comprising:
administering to an individual in need of such treatment an effective amount of Nrg-1β1.

According to another aspect of the invention, there is provided a method of treating multiple sclerosis comprising:
administering to an individual in need of such treatment an effective amount of Nrg-1β1.

As will be appreciated by one of skill in the art, an “effective amount” in regards Nrg-1β1 is an amount that is sufficient to improve and/or ameliorate and/or lessen the severity of one or more symptoms associated with multiple sclerosis, for example, dizziness, fatigue, pain, sensory impairment, numbness, tingling, tremors and weakness. Other symptoms associated with multiple sclerosis will be known to those of skill in the art.

The effective amount of Nrg-1β1 may be administered on a dosage regimen or schedule. For example, the Nrg-1β1 may be administered daily. As will be appreciated by one of skill in the art, “daily” does not necessarily mean every day, but may mean for example, 6 out of 7 days, 5 out of 7 days, 4 out of 7 days or the like. As discussed herein, we determined daily and continuous administration (throughout the disease course) of Nrg-1β1 is required for neurological recovery as short-term dosing did not render desirable therapeutic recovery. That is, in some embodiments, the effective amount of Nrg-1β1 is administered until symptoms have abated and/or until Nrg-431 levels, that is, Nrg-1β1 blood or plasma levels, have stabilized.

In some embodiments, the administration of the effective amount of Nrg-1β1 accomplishes one or more of the following: prevents activation, expansion and/or infiltration of leukocytes into the CNS tissue; increases plasma levels of Nrg-1β1; delay onset of symptoms associated with multiple sclerosis; reduce severity of symptoms associated with multiple sclerosis; suppress monocyte infiltration; foster a phenotype shift in CD11b+microglia and macrophages towards anti-inflammatory “M2”-like phenotype with a concomitant decrease in pro-inflammatory “M1”-like cells; and amend or restore reduced levels of Nrg-1 in the blood and in the CNS. In some embodiments of the invention, an “effective amount” is determined for example as the lowest amount administered to an individual in need of such treatment, for example, a human, that has at least on of the “effects” listed above.

As will be appreciated by one of skill in the art, such “an effective amount” can be determined by routine experimentation, using means known by those of skill in the art.

In some embodiments of the invention, the individual who is in need of such treatment is a pre-symptomatic individual who is at risk of developing MS. These individuals at risk can be determined by family history (persons with family members/close relatives having MS are at higher risk) or by virtue of having an MS associated infection, such as, for example, but by no means limited to, Epstein Barr virus.

Alternatively, the individual at risk is an individual who has plasma levels of Nrg-1β1 that are below a threshold level. As discussed herein, the threshold level may be a threshold value below which an individual is considered to have low or reduced levels of Nrg-1β1. In some embodiments, this threshold level may be determined from a healthy individual. The healthy individual may be an individual of similar age and general condition as the individual in need of such treatment. In some embodiments, the healthy individual or corresponding healthy individual also has no known health conditions and has not been diagnosed with any autoimmune diseases. Alternatively, the threshold level may represent a value determined from a plurality of individuals. In some embodiments, the threshold level of Nrg-1(3l is approximately or about 50% or less than that of a corresponding healthy individual, as defined above. As used herein, “about” or “approximately” in regard Nrg-1β1 levels, for example, Nrg-1β1 blood or plasma levels may represent plus or minus 10% of 50%, that is, between of the Nrg-1β1 blood or plasma levels of the corresponding healthy individual.

In some embodiments, the effective amount is between about 0.25 to about 10 μg Nrg-1β1 per kg body weight of the individual. In other embodiments, the effective amount is between about 1 to about 5 μg Nrg-1β1 per kg body weight of the individual. In yet other embodiments, the effective amount is between about 2 to about 3 μg Nrg-1β1 per kg body weight of the individual.

As discussed herein, administration of Nrg-1β1 has been demonstrated as a treatment for multiple sclerosis at various stages of the disease, for example, at onset (early stage of MS, first clinical presentation), peak of the disease (highest severity of disease in EAE model), and delayed (4-days after the peak of the disease in EAE). As such, it has been demonstrated that Nrg-1β1 is effective in MS patients at different stages of disease.

In some embodiments, the Nrg-1β1 is co-administered with a known or second medicament for treating multiple sclerosis, for example but by no means limited to interferon f31, glatiramer acetate, fingolimod, or alemtuzumab. It is of note that other suitable medicaments will be known to those of skill in the art. As discussed herein, these known medicaments can be combined with Nrg-1β1 for improved outcomes due to a multi-targeted approach. That is, although Nrg-1β1 can promote recovery from neurological deficits on its own, a combinatorial approach with other existing treatments will have synergistic or additive effects. It is of note that the Nrg-1β1 does not necessarily need to be co-administered with the second medicament for treating multiple sclerosis at exactly the same time, but may be “co-administered” in that both medicaments are administered individually to the individual over the same period of time, that is, over the same schedule or regimen.

According to an aspect of the invention, there is provided a method of prophylactically treating multiple sclerosis comprising:
administering to an individual in need of such treatment an effective amount of Nrg-1β1.

In these embodiments, an individual in need of such treatment is an individual who has not yet been diagnosed with multiple sclerosis but is at a higher risk of developing multiple sclerosis, for example, based on known risk factors and/or based on Nrg-1β1 blood or plasma levels, as discussed above. Specifically, as will be appreciated by one of skill in the art, individuals with lower Nrg-1β1 blood or plasma levels compared to a corresponding healthy control may be at greater risk of developing multiple sclerosis and/or may benefit greater from administration of an effective amount of Nrg-1β1, as discussed herein.

As will be appreciated by one of skill in the art, Nrg-1β1 can not be administered prophylactically to general population because over-stimulation of signaling pathways is associated with other diseases, such as, for example, cancer. Thus, only individuals at higher risk of developing MS (as described above) should be considered for prophylactic treatment.

In these embodiments, the individual in need of such treatment is an individual who has Nrg-1β1 blood or plasma levels approximately or about 50% compared to Nrg-1β1 blood or plasma levels of a corresponding healthy control.

According to another aspect of the invention, there is provided use of Nrg-1β1 for treatment or prophylactic treatment of multiple sclerosis.

According to another aspect of the invention, there is provided use of Nrg-1β1 for preparation of a medicament for treatment or prophylactic treatment of multiple sclerosis.

According to another aspect of the invention, there is provided Nrg-1β1 for treatment or prophylactic treatment of multiple sclerosis.

According to another aspect of the invention, there is provided Nrg-1β1 for preparation of a medicament for treatment or prophylactic treatment of multiple sclerosis.

As discussed herein, Nrg-1β1 can be administered prophylactically, symptomatically, acutely and/or chronically.

According to another aspect of the invention, there is provided a method for determining if an individual is a candidate for prophylactic treatment for multiple sclerosis with Nrg-1β1 comprising:
measuring an Nrg-1β1 level in a sample from the individual,
wherein, if the Nrg-1β1 level is below a threshold value, the individual is a candidate for Nrg-1β1 treatment.

According to another aspect of the invention, there is provided a method for determining if an individual is a candidate for further assessment for multiple sclerosis comprising:
measuring an Nrg-1β1 level in a sample from the individual,
wherein, if the Nrg-1β1 level is below a threshold value, the individual is further assessed for multiple sclerosis using clinical criteria for diagnosis (neurologic assessments, MRI etc). In case of negative clinical diagnosis, individuals would be scheduled for regular follow-up tests and monitored closely for 2-5 years.

In some embodiments, the sample is a blood sample, a plasma sample, whole blood, blood plasma, blood serum or a cerebrospinal fluid (CSF) sample.

As discussed herein, the threshold level may be a threshold value below which an individual is considered to have low or reduced levels of Nrg-1β1. In some embodiments, this threshold level may be determined from a healthy individual. The healthy individual may be an individual of similar age and general condition as the individual in need of such treatment. Alternatively, the threshold level may represent a value determined from a plurality of individuals. In some embodiments, the threshold level of Nrg-1β1 is approximately or about 50% or less than that of a corresponding healthy individual, as defined above. As used herein, “about” or “approximately” in regard a Nrg-1β1 level, for example, Nrg-1β1 blood or plasma levels may represent plus or minus 10% of 50%, that is, between 45-55% of the Nrg-1β1 blood or plasma levels of the corresponding healthy individual.

In some embodiments of the invention, the individual who is a candidate for treatment is administered an effective amount of Nrg-1β1 on a dosage schedule or regimen.

For example, the Nrg-1β1 may be administered daily. As will be appreciated by one of skill in the art, “daily” does not necessarily mean every day, but may mean for example, 6 out of 7 days, 5 out of 7 days, 4 out of 7 days or the like. As discussed herein, we determined daily and continuous administration (throughout the disease course) of Nrg-1β1 is required for neurological recovery as short-term dosing did not render desirable therapeutic recovery.

As will be apparent to one of skill in the art, the individual who is a candidate for treatment may be a pre-symptomatic individual who is at risk of developing MS. These individuals at risk can be determined by family history (persons with family members/close relatives having MS are at higher risk) of MS associated infections (Epstein Barr virus).

In some embodiments, the individual who is a candidate may be an individual who is showing other signs and/or symptoms of potentially having multiple sclerosis, such as for example but by no means limited to brain lesions as detected by MRI.

According to another aspect of the invention, there is provided a method for determining if treatment of an individual for multiple sclerosis is successful comprising:
taking a first sample from the individual;
measuring a first Nrg-1β1 level in the first sample from the individual,
then administering a treatment for multiple sclerosis to the individual for a period of time;
after said period of time, taking a second sample from the individual and measuring a second Nrg-1β1 level in the second sample from the individual; and
comparing the first Nrg-1β1 level to the second Nrg-1β1 level, wherein if the second Nrg-1β1 level is higher that the first Nrg-1β1 level, the treatment is successful.

The treatment for multiple sclerosis may be selected from the group consisting of Nrg-1β1, interferon β1, glatiramer acetate, fingolimod, alemtuzumab or other suitable medicaments known in the art.

The invention will now be further explained and/or elucidated by way of examples; however, the invention is not necessarily limited to and/or by the examples.

### Example 1—Dysregulated Levels of Nrg-1β1 Protein is Detected in Plasma, Spleen and Spinal Cord of EAE Mice and Precede Disease Onset

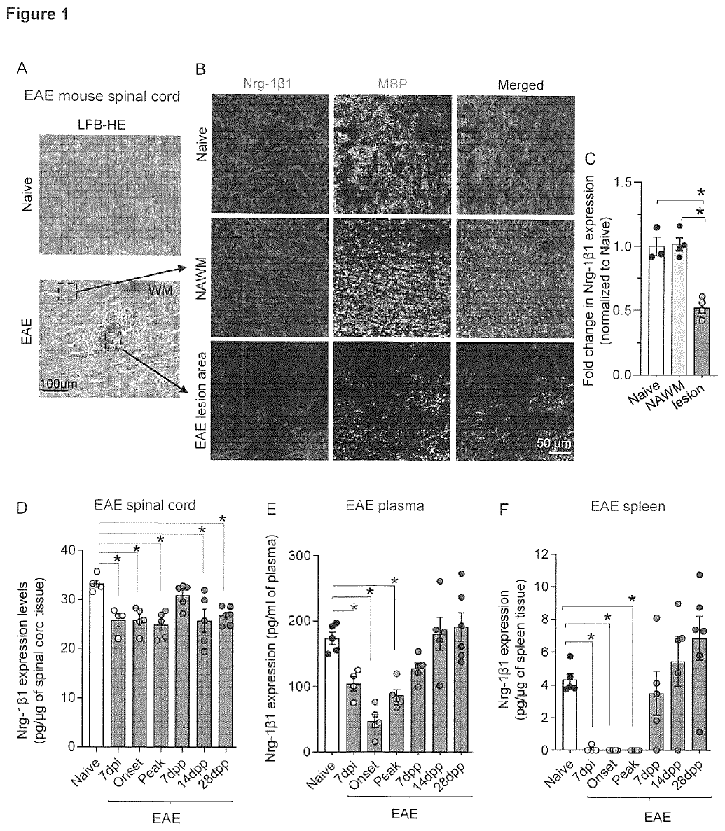

We conducted an in-depth investigation on Nrg-1β1 protein expression pattern in the spinal cord and peripherally in plasma and spleen of MOG35-55 induced EAE mice Immunohistological characterization of spinal cord lesions in EAE mice confirmed that Nrg-1β1 expression was significantly diminished within EAE demyelinating lesions (FIG. 1A-C). Nrg-1β1 immunofluorescence intensity measurement within the EAE lesions showed 48% reduction in Nrg-1β1 levels as compared to normal appearing white matter (NAWM) and naïve spinal cord tissue sections (FIG. 1C). These findings provide the first evidence that dysregulation of Nrg-1β1 protein is a characteristic of EAE lesions of the spinal cord. Since Nrg-1 is primarily expressed by neurons/axons and oligodendrocytes, and to a lesser extent by astrocytes [11, 21, 29], we asked whether decline in Nrg-1β1 within EAE lesions of white matter is attributed to loss of these cell types as the result of EAE. Our quantitative immunohistological assessment revealed 75% and 63% reduction in axonal and oligodendrocyte cell density, respectively, in EAE lesions as compared to naïve tissue and adjacent NAWM area in the same EAE mouse. On the contrary, immunofluorescence intensity measurement for astrocyte marker GFAP was significantly increased (60%) within the EAE lesions compared to naïve tissue and adjacent NAWM area in the same EAE mouse. Thus, these data suggest a positive correlation between diminished levels of Nrg-1β1 and reduced axonal and oligodendrocyte cell densities within the EAE lesions.

We also conducted a time-point ELISA analysis for Nrg-1β1 protein levels in the spinal cord of EAE mice and found a significant downregulation (22%) at the pre-symptomatic phase [7 days post-induction (dpi)], as compared to the baseline of Nrg-1β1 expression in the spinal cord of normal non-EAE mice (FIG. 1D). The decline in Nrg-1β1 protein levels persisted at the onset (12 dpi, 22%), peak of the disease (16 dpi, 25%), 14 days post peak (23%) that lasted chronically until 28 days post peak (dpp) (20%). However, at 7 dpp, there was a modest recovery in Nrg-1β1 levels by 18% as compared to the EAE peak, which could be possibly due to stochastic variations in samples. We further determined whether there is a correlation between peripheral levels of Nrg-1β1 in plasma and spleen with disease onset and progression. Interestingly, Nrg-1β1 level was significantly reduced in both plasma and spleen of EAE mice during the course of the disease at pre-symptomatic (7 dpi), onset (10-12 dpi) and peak (14-16 dpi), as compared to naïve animals (FIG. 1E-F). In plasma, Nrg-1β1 was significantly declined at 7 dpi (40%), onset (73%) and peak (50%) of EAE (FIG. 1E). In spleen tissue, the magnitude of Nrg-1β1 depletion was even more pronounced, as it was barely detectable at pre-symptomatic, onset and peak of EAE as compared to naïve mice (FIG. 1F). Nrg-1β1 levels were moderately recovered in plasma and spleen starting 7 dpp until 28 dpp, the last time point of our analyses (FIG. 1E-F). To confirm the overall integrity of protein in EAE plasma and tissue samples, we used serum albumin (BSA) and GAPDH as housekeeping proteins, respectively. ELISA assessment showed no significant change the levels of serum albumin (BSA) in the plasma or GAPDH in spleen and spinal cord lysates of EAE samples across various timepoints as compared to naïve samples, confirming that changes in Nrg-1β1 protein expression is a biological event related to EAE pathology. Collectively, these findings underscore a strong correlation between EAE pathogenesis and progression and Nrg-1β1 downregulation within the CNS and peripherally in plasma and spleen.

### Example 2—Nrg-1β1 Treatment Reduces Disease Severity in the EAE Mice with an Extended Therapeutic Time Window

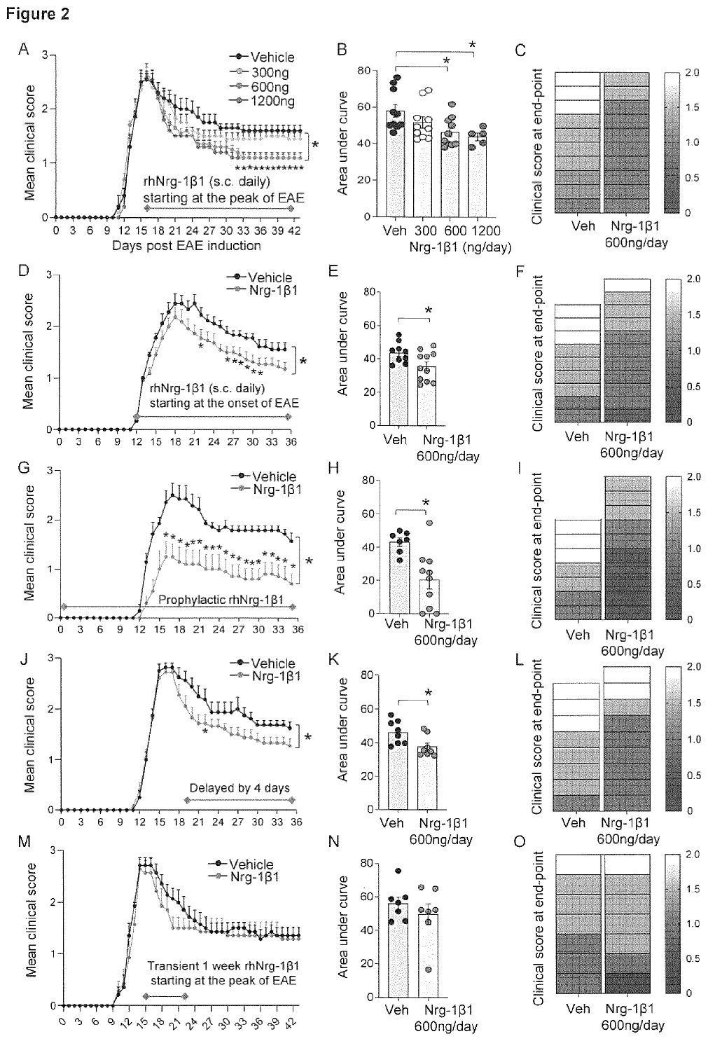

We next sought to determine whether CNS and systemic downregulation of Nrg-1β1 in EAE may have any functional ramifications on disease progression and severity. To this end, we systemically administered human recombinant Nrg-1β1 to EAE mice through daily subcutaneous injections. We performed systemic intervention as a clinically relevant strategy and the notion that Nrg-1β1 was declined both peripherally and in the spinal cord. Of note, Nrg-1β1 is an approximately 8 kDa peptide containing the bioactive EGF-like domain that is essential for activation of Nrg-1 signaling. Importantly, previous pharmacokinetic studies with similar peptide (8 kDa) confirmed that Nrg-1β1 peptide can readily pass the blood-CNS-barrier by saturable, receptor-mediated transport and enter CNS tissue [30]. We first performed a dose efficacy study with different concentrations of Nrg-1β1 peptide delivered at 300 ng, 600 ng and 1200 ng per day. To simulate the common clinical management of MS, we started Nrg-1β1 therapy once an EAE mouse reached peak of the disease (around day 14-16 post EAE induction, clinical score of 2.5-3 on a 5-point scale). EAE animals received daily treatment until 42 dpi. Control group received 0.1% BSA in saline, vehicle for Nrg-1β1, in the same manner. Daily clinical assessments by two experimenters blinded to animal treatments showed improved functional recovery in Nrg-1β1 treated EAE mice in a dose dependent manner (FIG. 2A). At the end point (42 dpi), the lower dose 300 ng/day dose did not induce any beneficial effects on disability score compared to vehicle treated EAE mice, suggesting that this dose did not reach the therapeutic threshold of Nrg-1β1 peptide that is required to induce significant improvements in EAE clinical scores. At the same timepoint, we found that both 600 ng and 1200 ng daily dose of Nrg-1β1 significantly and comparably improved functional recovery (26%), suggesting the ceiling effect was reached with 600 ng dose (FIG. 2A). Of note, previous pharmacokinetic studies by Kastin and colleagues has shown that radioactively labeled Nrg-1β1 peptide is relatively stable in mouse for 10 min after intravenous injection, and can cross the blood-CNS barrier by saturable, receptor-mediated transport and enters the parenchyma of brain and spinal cord [30]. Thus, it is plausible that beyond certain dose (e.g. 600 ng used in our study), the receptor-mediated transport for Nrg-1β1 becomes saturated and the therapeutic efficacy of the peptide reaches its peak with no further improvement in neurological scores. Based on the daily clinical scores, we also calculated the area under the curve as representative of cumulative disease burden for each animal. Similarly, our analysis showed a significant reduction in cumulative disease burden in EAE animals that received 600 ng (21%) and 1200 ng/day (26%) of Nrg-1β1 as compared to the vehicle treated EAE mice (FIG. 2B). Moreover, heat-map plot for the clinical score of each individual mouse at the end-point for vehicle and Nrg-1 (600 ng/day) treated group showed a significantly reduced disability score in Nrg-1β1 treated EAE animals compared to vehicle treated EAE mice (FIG. 2C). On the basis of this dosing study, we employed 600 ng/day (30 μg/kg/day) as an effective dose for Nrg-1β1 in all subsequent EAE studies.

Since our findings showed Nrg-1β1 level is markedly reduced early in EAE development, we asked whether restoration of Nrg-1β1 would ameliorate EAE severity and progression when treatment is administered at the onset of the EAE symptoms (day 12) or prophylactically at the time of EAE induction. Our long-term longitudinal evaluation for 36 dpi showed Nrg-1β1 treatment starting at the EAE clinical onset significantly reduced the cumulative burden of disease (21%) when compared to vehicle treatment (FIG. 2D-F). Prophylactic Nrg-1β1 treatment at the time of EAE induction more pronouncedly improved neurological disability (55% reduction) and cumulative disease burden (53%) in EAE mice and also delayed EAE progression (FIG. 2G-I). Collectively, these findings suggest that dysregulation of Nrg-1β1 has functional ramifications in the pathogenesis of EAE.

We extended our therapeutic studies to assess whether Nrg-1β1 therapy would be therapeutically beneficial if administrated in a delayed fashion after the peak of EAE. Interestingly, Nrg-1β1 therapy also attenuated the severity of EAE disability (21%) when it was delayed to 4 dpp compared to the clinical disability of vehicle treated animals at the endpoint (FIG. 2J-L). We further evaluated the necessity of sustained administration of Nrg-1β1 therapy for EAE recovery. To this end, we administered Nrg-1β1 treatment transiently for 7-days, starting at peak of the disease and assessed neurological recovery of EAE animals until 42 dpi. Interestingly, 7-day short-term treatment of Nrg-1β1 did not improve EAE-induced neurological deficits (FIG. 2M-O). Taken together, these observations suggest that dysregulation of Nrg-1β1 has significant implications in EAE onset and progression. Importantly, our therapeutic studies suggest that Nrg-1β1 treatment offers an extended therapeutic time window in EAE and can exert beneficial effects under different treatment paradigms. However, continuous administration of Nrg-1β1 appears to be critical for its beneficial effects in improving functional recovery in EAE.

### Example 3—Nrg-1β1 Ameliorates EAE by Limiting Leukocytes Infiltration and Inflammation Foci

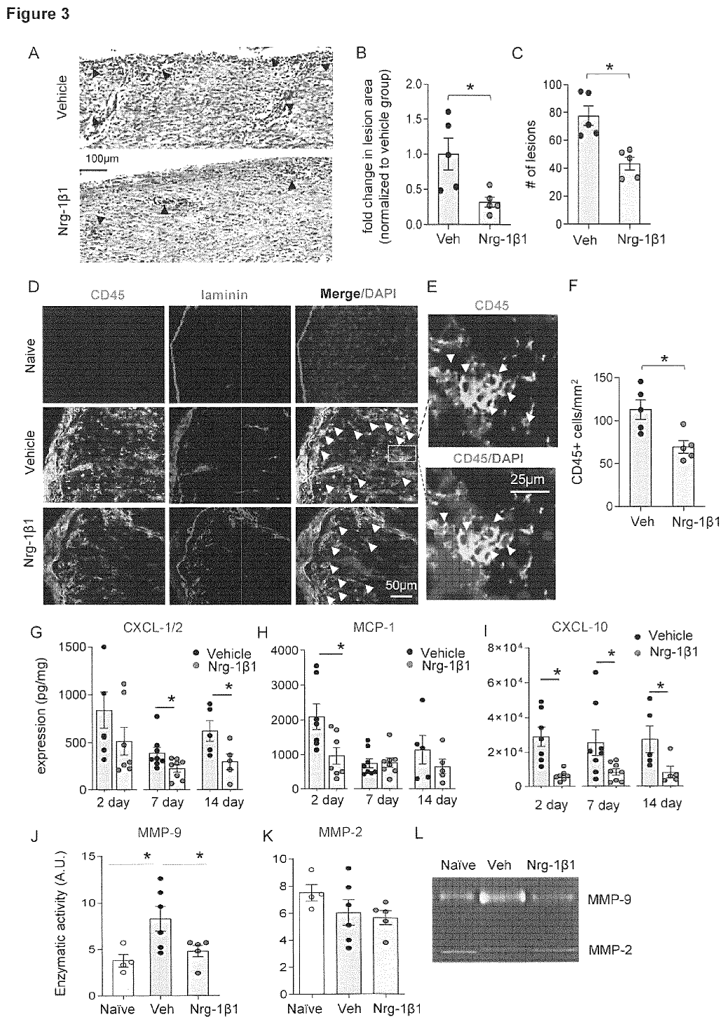

To unravel the potential mechanisms by which Nrg-1β1 treatment improves the neurological outcomes of EAE, we performed an array of histopathological, cellular and molecular analyses. Our overall histopathological analysis of LFB-HE stained spinal cord tissue after 2 weeks of treatment showed that Nrg-1β1 treatment significantly reduced the number of lesions (44%) and lesion area (60%) in the EAE mice as compared to the vehicle group (FIG. 3A-C). EAE lesions were identified by increased cellularity indicative of inflammatory infiltration. To confirm whether reduced EAE lesion size reflected a decrease in leukocyte infiltration, we employed immunostaining for CD45 (a general leukocytes marker) and laminin (marking vasculature and perivascular regions) in EAE lesions (FIG. 3D-E). We observed a considerable reduction in density of CD45+ cells in perivascular cuff and EAE lesions in the spinal cord. Our quantification of CD45+/DAPI+ cells within EAE lesions showed a significant reduction (38%) in infiltrating leukocytes in Nrg-1β1 treated group in relation to vehicle treated animals (FIG. 3F). To further unravel the underlying mechanisms by which Nrg-1β1 inhibits leukocyte infiltration into the spinal cord, we studied the expression pattern of known chemokines involved in this process by utilizing electro-chemiluminescence based multiplex ELISA at 2, 7 and 14 dpp. These timelines represent early and delayed leukocyte response in EAE progression. We found that Nrg-1β1 treatment modulates key chemokines involved in recruitment of neutrophils (CXCL1/2, Keratinocyte chemoattractant KC/human growth-regulated oncogene KC-GRO), monocytes (MCP-1, monocyte chemotactic protein-1) and T cells (CXCL10, Interferon-y-Inducing Protein-10) (FIG. 3 G-I). For CXCL1/2, we detected a significant reduction (42% and 52%) with Nrg-1β1 at 7 and 14 dpp, respectively, compared with vehicle treated EAE animals (FIG. 3G), while MCP-1 was significantly reduced (54%) at earlier time-point (2 dpp) and showed only modest effects at 7 and 14 dpp (FIG. 3H). Interestingly, Nrg-1β1 treatment significantly reduced CXCL10 (>65%) at all the examined time points (FIG. 3I). These findings indicate that limiting leukocyte infiltration into the CNS tissue is one immunomodulatory mechanism by which Nrg-1β1 attenuates EAE severity.

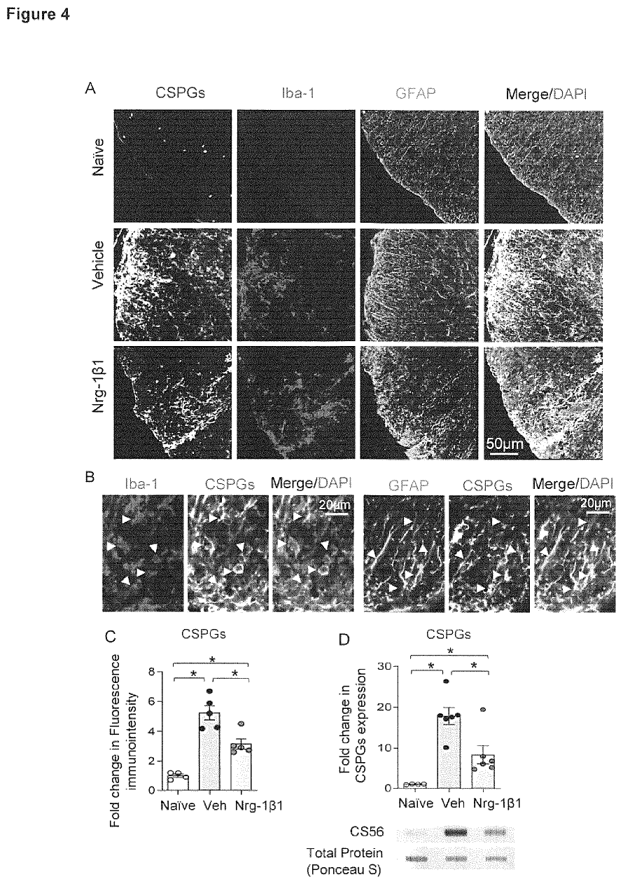

In EAE, matrix metalloproteinases (MMPs), in particular MMP-9, disrupt the integrity of blood-CNS barrier and thereby facilitate leukocytes infiltration into the CNS tissue [31]. Thus, we asked whether Nrg-1β1treatment influences MMP activity in EAE. Through gelatin zymography, we assessed the enzymatic activity of MMP-2 and MMP-9 within the spinal cord tissue. We demonstrate that Nrg-1β1 treatment significantly attenuated the EAE-induced increase in MMP-9 activity by 42% (FIG. 3J, L). However, we found no apparent changes in MMP-2 activity as the result of EAE (FIG. 3K-L). Recent studies have implicated chondroitin sulfate proteoglycans (CSPGs) in pathogenesis of EAE and MS [32-34]. Upregulation of CSPGs after EAE is shown to promote accumulation of leukocytes in the perivascular cuff and facilitate their infiltration into the EAE lesions [32]. Our immunohistochemical analysis of EAE lesions confirmed co-localization of CSPGs with microglia and macrophages (Iba-1+) as well as astrocytes (GFAP+), as expected (FIG. 4A-B). However, activated microglia and macrophages seemed to show a greater degree of co-localization with CSPGs in EAE lesions than astrocytes. Our quantitative immunofluorescence intensity analysis of CSPGs showed that Nrg-1β1 treatment reduced the EAE induced upregulation of CSPGs in the spinal cord by 40% (FIG. 4A, C). These results were corroborated with our complementary slot blot analysis of CSPGs (FIG. 4D). Our findings indicate that availability of Nrg-1β1 attenuates leukocyte infiltration in EAE through several mechanisms including modulation of chemokines, MMP-9 and CSPGs.

### Example 4—Availability of Nrg-1β1 Positively Regulates Innate Immune Response in EAE

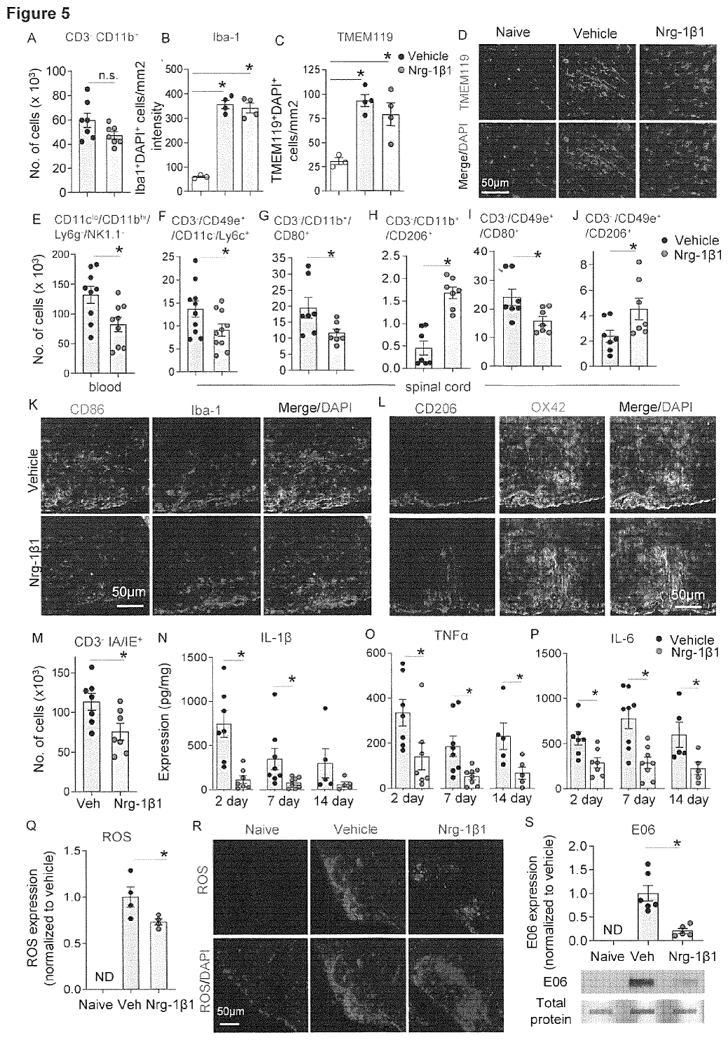

CNS microglia and monocyte-derived macrophages are components of the innate immune response that play a pivotal role in EAE pathogenesis [35-37]. We sought to determine whether Nrg-1β1 treatment influences the response of microglia and monocyte-derived macrophages. Our flow cytometry of spinal cord tissue at 2 dpp and 7 dpp (FIG. 5) showed no significant change in the overall presence of CD3−/CD11b+ population between Nrg-1β1 and vehicle treated EAE mice suggesting the number of microglia and macrophage remains relatively unchanged under Nrg-1β1 therapy (FIG. 5A). This was confirmed by complementary immunohistochemical analysis of Iba-1+ microglia and macrophages in the spinal cord of EAE mice, which also remained unchanged (FIG. 5B). To differentially examine the effect of Nrg-1β1 treatment on resident microglial, we performed cell count for the microglia specific marker, TMEM119 and found no significant difference in the number of microglia in EAE lesions of Nrg-1β1 versus vehicle treated mice (FIG. 5C-D). Next, we specifically examined circulating monocytes and monocyte derived macrophages by flow cytometry in the blood (CD3−/CD11clo/CD11bhi/Ly6g−/NK1.1−) and spinal cord tissue (CD3−/CD49e+/CD11c−/Ly6c+), respectively. Intriguingly, we detected a significant reduction in both circulating (46%) and infiltrating (28%) monocytes with Nrg-1β1 treatment at 7 dpp (FIG. 5E-F), while they remained unaltered at 2 dpp. These findings suggest that the overall reduction in leukocytes that we detected in EAE lesions after 7 days of Nrg-1β1 treatment reflects, at least in part, its suppressive effects on monocyte expansion and/or infiltration into the CNS.

Since the phenotype of microglia and monocyte-derived macrophages has a significant impact on the neuroinflammatory landscape in EAE, we next studied whether Nrg-431 treatment modulates the immune properties of these cells in the spinal cord of EAE mice. Our flow cytometry assessment identified a significant reduction (40%) in CD3−/CD11b+/CD80+ pro-inflammatory “M1” type microglia and macrophages with Nrg-1β1 treatment as compared to vehicle group at 7 dpp. However, there was no significant change at 2 dpp time-point analysis (FIG. 5G). This response was also accompanied by a striking increase in CD3−/CD11b+/CD206+ anti-inflammatory “M2”-like microglia and macrophages under Nrg-1β1 treatment at both 2 dpp (317%) and 7 dpp (274%) (FIG. 5H). We further assessed whether Nrg-1β1 influences the phenotype of infiltrating monocytes in the EAE spinal cord. Flow cytometry indicated Nrg-1β1 treatment significantly decreased the number of monocyte derived “M1”-like macrophages (CD3−/CD49e+/CD80+) by 34% at 7, while promoting “M2”-like macrophages (CD3−/CD49e+/CD206+) at both 2 dpp and 7 dpp (28% and 90%, respectively) (FIG. 5I-J). However, there was no change in monocyte derived “M1”-like macrophages with Nrg-1β1 treatment at 2 dpp time-point. Our complementary immunohistochemistry also verified this M1/M2 phenotype shift in the spinal cord of Nrg-1β1 treated EAE mice (FIG. 5K-L). Since microglia and macrophages also act as antigen presenting cells (APCs) in EAE, we studied the effects of Nrg-1β1 on their phenotype in this context. Interestingly, while Nrg-1β1 treatment significantly reduced (33%) the overall number of CD3−/IA/IE+ APCs in EAE lesions, it did not alter CD3−/CD11b+/IA/IE+ microglia/macrophage APCs (FIG. 5M).

Cytokine release profile of immune cells reflects their functional impact on the neuroinflammatory response in EAE. Thus, we also conducted a time course analysis of some key cytokines associated with pro-inflammatory “M1”-like cells in the spinal cord of EAE mice using multiplex mesoscale platform. We found that Nrg-1β1 treatment dramatically reduced the release of interleukin (IL)-1β at 2- and 7-day post EAE peak. IL-6 and tumor necrosis factor alpha (TNF-α) levels were also declined significantly in the spinal cord after Nrg-1β1 treatment at all examined time-points (2, 7, 14 dpp), as compared to the vehicle group (FIG. 5N-P). However, spinal cord levels of the anti-inflammatory cytokine IL-10 remained unchanged with Nrg-1β1 treatment at all time points.

Reactive oxygen species (ROS) derived from macrophages are involved in EAE and MS pathogenesis [38-40]. Thus, we asked whether the decrease in “M1” macrophages would be associated with reduced ROS levels in the spinal cord tissue. We assessed ROS levels in the spinal cord of EAE mice with red fluorescent ethidium signal intensity generated by oxidation of dihydroethidium (DHE). EAE expectedly induced a robust increase (73%) in the basal levels of ROS, which was significantly reduced in Nrg-1β1 treated EAE mice (27%) (FIG. 5Q-R). We also asked whether reduction in “M1” macrophages and ROS levels may attenuate EAE induced lipid peroxidation during oxidative stress. Slot blot analysis of oxidized lipids marker, E06, showed high levels of lipid peroxidation in the EAE mice at 14 dpp, as compared to its non-detectable levels in non-EAE naïve animals. Nrg-1β1 treatment remarkably attenuated oxidized lipids (80%) as compared to vehicle treated EAE mice (FIG. 5S). Collectively, our findings indicate that Nrg-1β1 regulates monocyte expansion and infiltration peripherally in EAE and fosters a phenotype in macrophages and microglia in the CNS that supports resolution of the pro-inflammatory landscape in EAE mice.

### Example 5—Nrg-1β1 Mitigates T Helper 1 Response Directly and Indirectly by Modulating Macrophages

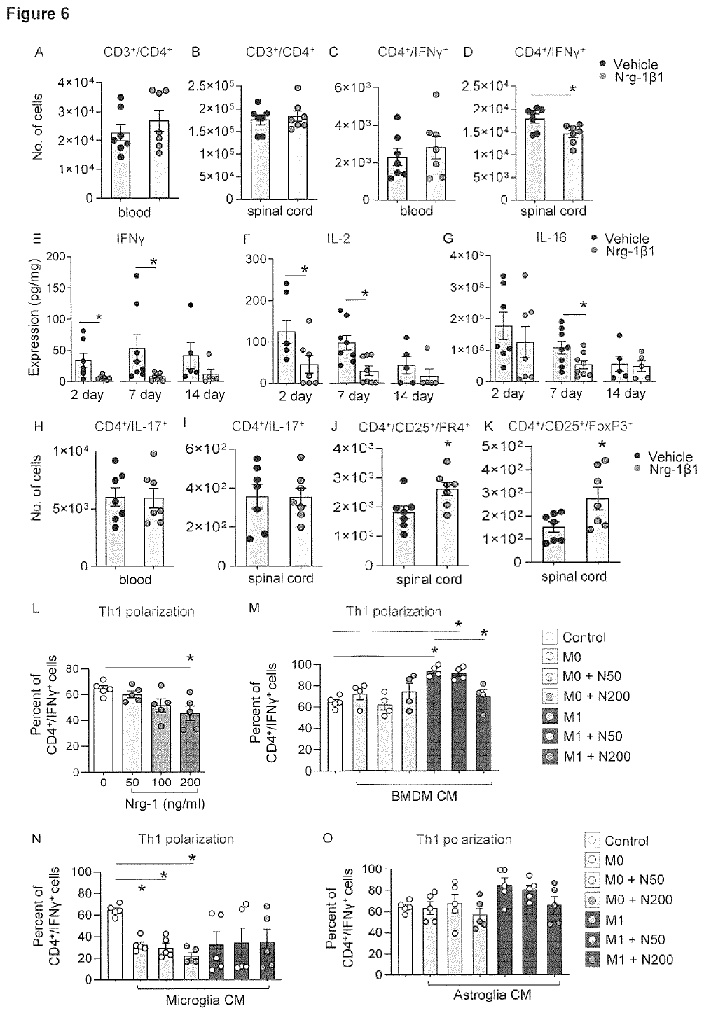

T cell-triggered autoimmunity is a major mechanism of EAE and MS pathogenesis [41]. Therefore, we next investigated whether Nrg-1β1 modulates EAE pathogenesis and resolution by influencing T helper cell population. Flow cytometry at 2 and 7 dpp identified no change in total number of CD3+/CD4+ T cells in the blood or spinal cord of EAE mice suggesting Nrg-1β1 did not affect overall T cell expansion peripherally, nor their presence in the spinal cord (FIG. 6A-B). However,

Nrg-1β1 treatment appeared to influence T cell phenotype in EAE lesion as we detected a significant reduction (18.73%) in the T helper type 1 (Th1) interferon gamma cytotoxic cells (CD4+/IFNγ+) population in the spinal cord of EAE mice at 7 dpp time-point (FIG. 6C-D). Of note, the number of circulating Th1 CD4+/IFNγ+ cell population in the blood remained unaffected with Nrg-1β1 treatment, suggesting that the effects of Nrg-1β1 on T cell phenotype were more specific to the environment of EAE spinal cord (FIG. 6C). Nrg-1β1 effects were also accompanied by a significant reduction in the spinal cord levels of IFNγ+, IL-2 and IL-16 under Nrg-1 treatment at 2 dpp and/or 7 dpp (FIG. 6E-G). These cytokines are key mediators of Th1 cell differentiation and function in EAE and MS [42]. We also studied T helper 17 (Th17) response, another key driver of EAE pathogenesis [43]. However, flow cytometry of effector Th17 population (CD4+/IL-17+) showed that Nrg-1β1 treatment did not alter effector Th17 population in the blood or the spinal cord of EAE mice at 2 dpp and 7 dpp (FIG. 6H-I). In contrast, anti-inflammatory T regulatory cells (CD4+/CD25+/FR4+ and CD4+/CD25+/FoxP3+) were significantly elevated at 2 and 7 dpp as the result of Nrg-1β1 treatment (FIG. 6J-K). These EAE findings suggest that although availability of Nrg-1β1 does not suppress the overall recruitment of T cells peripherally or in the EAE lesions, it fosters a more balanced T cell response by suppressing effector Th1 response while promoting T regulatory populations.

Response and phenotype of T cells in EAE pathogenesis and recovery is highly influenced by their cross-talk with CNS innate immune cells (microglia, macrophages and astrocytes) [35, 36, 41, 43-46]. Since we found that Nrg-1β1 regulated T cell phenotype in the spinal cord but not in the blood, we asked whether it influenced T cell phenotype indirectly through its modulatory effects on astrocytes, microglia and/or monocyte derived macrophages in EAE lesions. Notably, our immunocytochemical assessments confirmed that microglia, macrophages and astrocytes express Nrg-1β1 ligand binding receptors, ErbB2 and ErbB4. To address this hypothesis, we performed in vitro studies. We polarized naïve CD4+ T cells under Th1 and Th17 polarization conditions and subject them to conditioned media (CM) from microglia, bone marrow derived macrophages (BMDM) or astrocytes under M0 (control) or M1 (IFNγ+LPS treated) conditions with or without Nrg-1β1 treatment. First, we demonstrate that direct treatment with Nrg-1β1 resulted in a reduction in the number of Th1 polarized cells (CD4+/IFNγ+) in a concentration dependent manner, as compared to the control condition (FIG. 6L). Then, we found that CM of M1 BMDM significantly increased Th1 population (45%), while M1 BMDM treated with Nrg-431 (200 ng/ml) decreased Th1 cells (26%). Treatment with Nrg-1β1 did not affect the effects of M0 non-activated BMDM cells on Th1 polarization. This demonstrates that availability of Nrg-1β1 can inhibit Th1 polarization by regulating the response of pro-inflammatory M1 macrophages (FIG. 6M). Interestingly, CM of activated M1 microglia or astrocytes CM did not result in any significant change in the population of Th1 polarized cells, suggesting a specific role for macrophages in promoting Th1 response (FIG. 6N-O). Our flow cytometry studies of Th17 polarized cells showed no changes in the number of Th17 effector cells (CD4+/IL-17+) neither under Nrg-1β1 nor BMDM or microglia CM, while astrocytes CM itself (both M0 and M1) significantly attenuated the generation of CD4+/IL-17+Th17 cells, although it was irrespective of Nrg-1β1 treatment. Interestingly, our in vitro flow cytometry revealed that Nrg-1β1 (100 ng and 200 ng/ml) also directly reduced (27-35%) Th1 effector cells (CD4+/IFNγ+) under Th17 polarization, confirming our finding in the EAE mice. Although CM of BMDM (both M0 and M1) did not affect the number of CD4+/IFNγ+ cells, CM from M0 and M1 microglia significantly attenuated these pro-inflammatory cells. However, this effect was irrespective of Nrg-1β1 treatment. In contrast, CM from activated astrocytes treated with Nrg-1β1 (200 ng/ml) significantly attenuated CD4+/IFNγ+ effector Th1 under Th17 polarization in comparison to CM of non-activated astrocytes treated condition. Taken together, these findings suggest that Nrg-1β1 primarily regulate the Th1 mediated inflammatory response in EAE directly, which appears to be directly and indirectly through modulation of monocytes/macrophages and to some extent astrocytes.

### Example 6—Proteomics Asserts the Impact of Nrg-1β1 on Modulating Pathways Involved in Immune Response and Lipid Oxidation in EAE

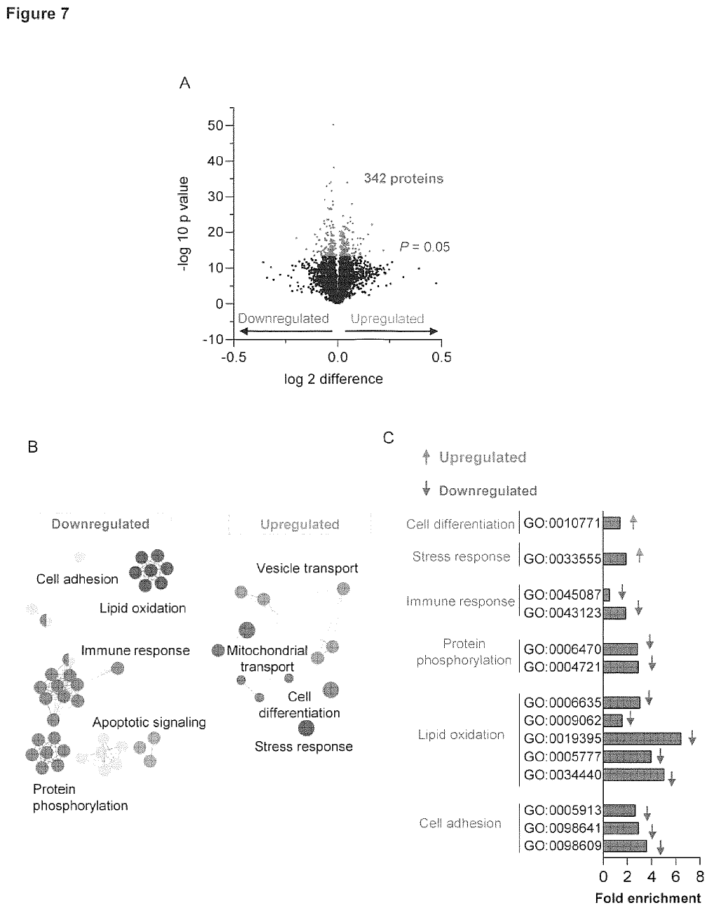

To further validate immune modulatory role of Nrg-1β1 treatment in the EAE, we performed LC-MS/MS based proteomics on the spinal cord tissue of EAE mice at 7 days post treatment; the time-point that Nrg-1β1 showed most of its significant regulatory effects. Comparing Nrg-1β1 and vehicle treated groups, we found 342 differentially expressed proteins as the result of Nrg-1β1 therapy (FIG. 7A). Pathway analysis of upregulated and downregulated proteins in Nrg-1β1 treated group with respect to vehicle group unveiled some of the key biological functions related to immune response, lipid oxidation, stress response, apoptotic signaling and mitochondrial/vesicle transport (FIG. 7B). A detailed database search through DAVID software corroborated these findings as immune response, lipid oxidation, cell adhesion and cell differentiation were some of the GO pathways, which significantly enriched in pathway analysis (FIG. 7C). To further elaborate on this bioinformatics data, we performed specific pathway analysis using ClueGo software. Reactome and GO database pathway analysis affirmed our ELISA and flow cytometry assessment suggesting the involvement of IL-1, IL-17 and TLR cascades in Nrg-1β1 mediated effects in EAE spinal cord tissue. Of note, Nrg-431 ameliorated the proteins/transcription factors associated with leukocyte trans-endothelial migration, chemokine mediated migration, leukocyte infiltration and macrophage-restricted adhesion molecules, confirmed our cellular assessment that showed availability of Nrg-1β1 inhibits leukocyte infiltration into the CNS during EAE pathogenesis. Interestingly, pathway analysis also affirmed the effects of Nrg-1β1 in attenuating lipid oxidation and fatty acid catabolic processes. Collectively, our immunohistochemical, flow cytometry, cytokine profiling and proteomics data indicate that Nrg-1β1 reduces monocyte extravasation from the periphery thereby abating the Th1 (IFNγ) mediated inflammatory response in the CNS of EAE mice, which results in reduced/delayed EAE symptoms and facilitates the recovery process.

### Example 7-Nrg-1β1 is Depleted in Active Demyelinating Plaques of MS Patients

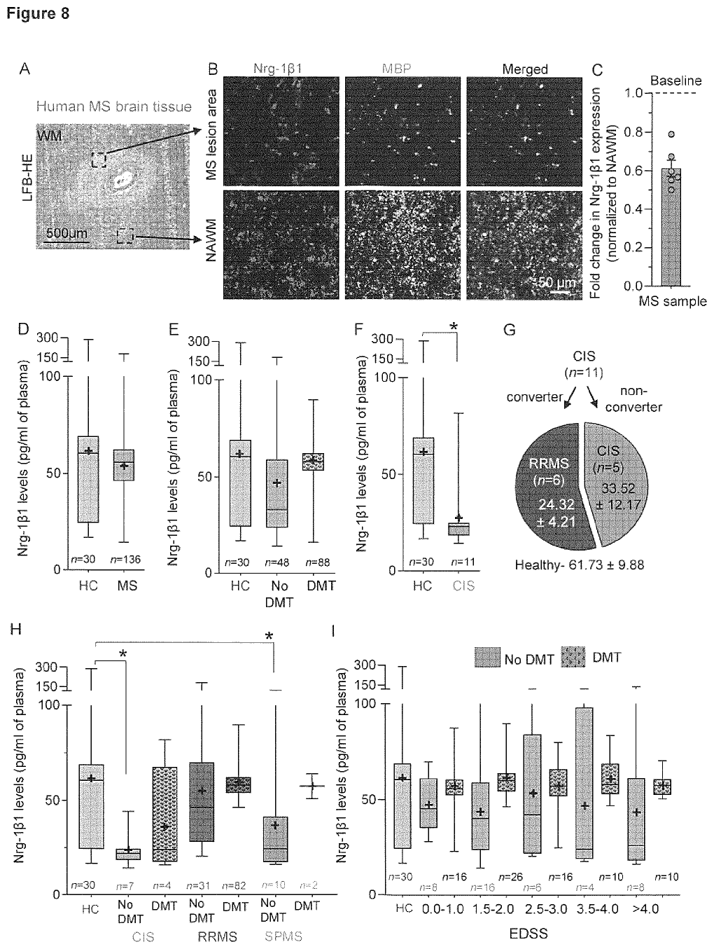

To validate the relevance of our EAE studies to MS pathophysiology, we investigated Nrg-1β1 protein expression in active demyelinating plaques of six MS brain samples. Using Luxol Fast Blue and hematoxylin eosin (LFB-HE), we first identified MS demyelinating plaques in the white matter (FIG. 8A). We further confirmed demyelinating lesions by reduced immunofluorescence intensity of myelin basic protein (MBP). Our immunohistochemical analysis showed a significant reduction in Nrg-1β1 expression levels within MS plaques as compared to the surrounding NAWM (FIG. 8B-C). To quantify and account for the variability among MS tissue samples, we normalized immunofluorescence intensity values for each lesion to NAWM adjacent area of the same sample. Collective analysis of all samples showed that Nrg-1β1 intensity levels was reduced within the MS plaques by 39% as compared to NAWM. These results provide evidence supporting a positive association between Nrg-1β1 downregulation and MS pathology (FIG. 8C).

### Example 8—Plasma Levels of Nrg-1β1 are Lower in Patients with Early MS and are Associated with Subsequent Progression to Relapsing-Remitting MS

We next determined whether downregulated levels of Nrg-1β1 is also detected peripherally in the plasma of MS individuals, as detected in the EAE mice. We analyzed Nrg-1β1 levels in plasma samples of MS and normal individuals. ELISA analysis included normal participants (N=30) and MS individuals of a patient cohort (N=136) presenting three major sub-types of MS including clinically isolated syndrome (CIS, N=11), relapsing remitting MS (RRMS, N=113) and secondary progressive MS (SPMS, N=12). Primary progressive MS (PPMS) type was not included in our analysis due to insufficient samples within the cohort. Our initial analysis comparing plasma level of Nrg-1β1 among normal individuals and all MS patients, regardless of their disease subtype, showed no significant difference (FIG. 8D). To ascertain whether Nrg-1β1 expression may vary under disease modifying treatments (DMTs) at the time of sample collection, we also compared Nrg-1β1 levels between DMTs users and non-users. Our analysis indicated no overall difference in Nrg-1β1 levels among all MS patients receiving DMTs as compared to patients who did not receive any treatment (FIG. 8E). The apparent lack of statistically significant difference in plasma levels of Nrg-1β1 in DMT users and non-user patients could be attributed to the smaller sample size and high variability within the group.

We next determined the plasma levels of Nrg-1β1 among different sub-types of MS. We first plotted Nrg-1β1 levels of MS patients grouped into respective clinical diagnosis for the disease and then sub-grouped on the basis of whether they were receiving any DMTs at the time of plasma collection. Intriguingly, we found significantly lower (>50%) plasma levels of Nrg-1β1 in CIS individuals, irrespective of receiving DMTs, in comparison to normal individuals (FIG. 8F). Importantly, 55% (6 out of 11) of the CIS individuals in this study developed RRMS during a median follow-up of 4 years of the onset of CIS. Of note, the CIS patients who developed RRMS also showed lower levels of Nrg-1β1 (61% reduction) in plasma, as compared to those who did not develop MS (46% reduction) until their last clinical visit (FIG. 8G). These initial findings provide first evidence suggesting that Nrg-1β1 is dysregulated in early phase of MS.

Next, we investigated the relationship between Nrg-1β1 plasma levels and DMTs among MS subtypes. Interestingly, Nrg-1β1 levels in CIS and SPMS individuals who did not receive DMTs were significantly reduced as compared to normal individuals. Nrg-1β1 levels of RRMS patients, both DMT and non-DMT receiving, were closer to normal individuals than CIS and SPMS individuals (FIG. 8H,). Furthermore, we examined whether Nrg-1β1 plasma levels correlate with the expanded disability status scale (EDSS) or number of years spent after MS diagnosis and DMTs for each individual (FIG. 8I). We did not find any statistically significant difference in our analysis due to high degree of variation, although there was an overall reduction in the levels of Nrg-1β1 in individuals who did not receive any DMTs with respect to EDSS score (FIG. 8I). Similarly, there was reduced expression of Nrg-1β1 during early years of MS with respect to EDSS score. However, the difference was not statistically significant as compared to healthy controls. Overall, the smaller sample size across the groups, higher variability in expression of Nrg-1β1 among MS patients and disparate stage of the disease plausibly led to non-significant outcomes of these analyses. Future studies with larger MS patient sample size and adequate representation of all stages of the disease are warranted to have conclusive evidence about the relation of Nrg-1β1 with disease progression and DMT administration.

### Materials and Methods

### Study Design

To evaluate the potential role of Nrg-431 in MS disease pathogenesis, we assessed protein levels of Nrg-1β1 in plasma, spleen and spinal cords of EAE mouse model. These observations were corroborated with postmortem MS brain tissue and plasma of cohort of MS patients. Further, we evaluated the therapeutic potential of recombinant human Nrg-1β1 (rhNrg-1β1) in the EAE mouse model. We employed various therapeutic time window including treatment administration at the peak, onset, prophylactically and post-peak. Of note, a clinical grade of rhNrg-1β1 has received approval from Food and drug Administration (FDA) for Phase II and III clinical trials for chronic heart failure, indicating its safety. To elucidate the underlying mechanisms, cytokine profiling, flow cytometry and proteomics were performed. Animals were randomly allocated to treatment groups. Observers were blinded to experimental groups during clinical score assessments. All the experimental procedures and assessments were performed in blinded manner. No animals or samples in any of the experiments were excluded from data analyses, unless specified otherwise.

### Animal Studies

All animal procedures and experimental protocols were approved by the Animal Ethics Care Committee of the University of Manitoba in accordance with the policies established in the guide for the care and use of experimental animals prepared by the Canadian Council of Animal Care. Mice were housed with a 12-hour light/dark cycle in standard plastic cages at 22° C. Drinking water and pelleted food were given ad libitum. For in vivo EAE studies, a total of 266 C57BL/6 female mice (8 weeks old) and for in vitro experiments, 10 C57BL/6 female mice (10 weeks old) and 25 C57BL/6 female pups (1-3 days old) were used. All animals were provided by Central Animal Facility, University of Manitoba, Canada.

### EAE Induction and Treatments

C57BL/6 female mice (8 weeks old) were provided by the Central Animal Facility of University of Manitoba, Canada. Mice were acclimatized for at 7 days prior to immunization with 100 μL (5014) of MOG 35-55 peptide in incomplete Freund's adjuvant (IFA) supplemented with 5 mg/mL heat-inactivated Mycobacterium tuberculosis H37Ra (Thermo Fisher Scientific). 50 μL emulsion was injected subcutaneously (s.c.) on either side of the tail base. 300 ng of Pertussis toxin (List Biological Laboratories) was injected intraperitonially (i.p.) on days 0 and 2 after MOG immunization. Daily monitoring of EAE mice was performed, and the mice were scored based on the degree of disability of tail and limbs on a scale of 5. EAE mice were randomly assigned to experimental groups: vehicle and Nrg-1β1. Animals in Nrg-1β1 group received daily s.c. injections of rhNrg-1β1 peptide (˜8 kDa) containing the bioactive epidermal growth factor (EGF)-like domain (Shenandoah Biotechnology, USA) at indicated doses. Vehicle animals received equivalent volume of 0.1% bovine serum albumin (BSA) in saline. Treatments were administered daily under different paradigms: at the time of EAE induction (prophylactically), at the onset of EAE symptoms (clinical score of 0.5), at the peak of the disease (clinical score of 2.5-3) or in delayed fashion at 4 days after reaching the peak. EAE mice in transient therapeutic paradigm received treatments for 7 days starting at the peak of the disease.

### Histology and Immunofluorescence Staining

At identified end-points, deeply anesthetized mice (isoflurane/propylene glycol; 40:60 v/v) were perfused with cold 0.1M of phosphate buffer saline (PBS) and 3.5% paraformaldehyde (PFA) for immunohistochemical analyses. Thoracic and lumbar spinal cord tissue was post-fixed in 10% sucrose in 3.5% PFA at 4° C. overnight, followed by cryoprotection in 20% sucrose for additional 24-48 hrs. Spinal cord tissue embedded in Tissue-Tek®-OCT (Electron Microscopy Sciences) was cut in serial sections of 16 μm thickness on a cryostat (Leica Biosystems) and stored at −80° C. until further use. EAE spinal cord sections.

For quantitative assessment of inflammatory EAE lesions, for each mouse 20 serial cross-sections of the spinal cord (with 500 μm interval) were stained with Luxol Fast Blue (LFB) and hematoxylin and eosin (HE) and imaged. Number of parenchymal inflammatory foci (lesions) per spinal cord section were quantified by a treatment-blinded examiner and plotted as sum of all lesions observed in 20 sections for each animal. For analysis of lesion area, inflammatory foci in the spinal cord parenchyma were identified in LFB-HE stained sections and quantified within each section using Image J software (NIH). The sum of the areas of all measured lesions within a cross-section was calculated and divided by total area of the spinal cord cross-section to normalize it for variation in spinal cord area. Average lesion area for each animal was then calculated as mean of lesion area from spinal cord cross-sections and represented as fold change in area as compared to vehicle group.

For immunofluorescence intensity assessments, three spinal cord cross-sections with lesion and at least 500 μm apart were selected from each animal. Frozen slides were air dried at room temperature (RT) and washed with PBS for 5 min followed by incubation with blocking solution (1% BSA, 5% non-fat milk, and 0.3% Triton X-100 in PBS) for 1 h at RT. Tissue sections were incubated with the primary antibody in blocking solution overnight at 4° C. Slides were washed in PBS, and incubated with either fluorescent Alexa 488-conjugated, Alexa 568-conjugated or Alexa 647-conjugated anti-mouse/rabbit/chicken/goat secondary antibodies (1:500; Invitrogen) as appropriate for co-labeling. The tissue sections were stained with the nuclear marker 4′,6-diamidino-2-phenylindole (DAPI), and cover-slipped with Mowiol mounting medium. Specificity of all antibodies was confirmed using both a negative control, omitting the primary antibody in our immunostaining protocol, and a positive control, testing the antibody on tissues or cell preparations known to express the target antigen. All samples were processed in parallel under the same condition and imaged using Zeiss Axiolmager M2 fluorescence microscope (Zeiss) under consistent setting.

### Imaris Quantification for Cell Counting

The Imaris software (Bitplane, Switzerland) was used to determine the cell count of DAPI+ cells within and around EAE lesions that were Iba-1+ or TMEM119+. Iba-1 and TMEM119 labelled areas of a cell were rendered as surfaces, and the nuclear marker DAPI was rendered as spots. The Xtension program in Imaris ‘surface close to spots’ was used to calculate the nuclei that were within 2 μm of Iba-1+ or TMEM119+ staining to provide the best possible count of the respective cell types.

### Detection of Reactive Oxygen Species (ROS)

ROS production was detected in EAE mice at end-point with intraperitoneal injection of dihydroethidium (DHE, 10 mg/kg) (Molecular Probes, Invitrogen) as described earlier [27]. The DHE is oxidized by reactive species within the cell, providing an index of the production of reactive species. Mice were euthanized 3 h after DHE injection and transcardially perfused as described above. Oxidized DHE signals were imaged after co-labelling with DAPI and immunofluorescence intensity was measured by image J (NCBI, MD) and expressed as fold change in mean gray value normalized to naïve mice.

### Cell Preparation and Flow Cytometry

For isolation of mononuclear cells from the spinal cord, entire spinal cord tissue was enzymatically digested with 2 mg/ml of Collagenase A (C5138, Sigma), 10U/ml Papain (P4762, Sigma), 1 mg/ml DNase I (D5025, Sigma) in Dulbecco's modified eagle media (DMEM) for 20 minutes at 37° C. The digested tissue was passed through 40 μm cell strainers to obtain single-cell suspensions, and Percoll gradient centrifugation was performed to obtain mononuclear cells. Peripheral mononuclear blood cells (PBMCs) were isolated by collecting blood through cardiac puncture in presence of Ethylenediaminetetraacetic acid (EDTA) as anticoagulant. After centrifugation at 600 g for 5 min, red blood cells (RBC) were removed from PBMCs by incubation in RBC lysis buffer for 5 min at room temperature (RT). PBMCs were washed twice with PBS followed by re-suspension in flow buffer (4% FCS, 0.05% Sodium azide in PBS). Intracellular staining was performed after cell permeabilization using the Fix/Perm Buffer Set (BD Biosciences, USA) according to the manufacturer's instructions. Isotype-matched controls and Fluorescence Minus One (FMO) controls were included in each staining. Viable cells were gated using Fixable Viability Stain 780 (565388, BD Biosciences). FACS data was acquired on the CytoFlex LX digital flow cytometry analyzer (Beckman Coulter, USA) and analyzed using FlowJo software.

### Immunoblotting for CSPGs and Lipid Peroxidation

Mouse lumbar spinal cord tissue was homogenized in NP-40 lysis buffer containing protease inhibitor cocktail (Sigma). Slot blotting was performed to detect the expression of chondroitin sulfate proteoglycans (CSPGs) and oxidized lipids with antibody against GAG portion of native CSPGs (clone CS-56, Sigma) and oxidized phospholipids (E06, Avanti, Millipore Sigma), respectively. 2-514 of protein of spinal cord tissue sample was blotted on a nitrocellulose membrane using Bio-Dot® slot blot system. The membrane was washed with 1% TBST followed by Ponceau S staining for total protein loading. The blot was blocked with 5% skim Milk and incubated with primary antibody in blocking solution for 1 hour at room temperature followed by incubation with secondary HRP antibody (1:4000; BioRad), followed by incubation in ECL immunoblotting detection reagents (FroggaBio, Canada). Immunoreactive bands were quantified with AlphaEaseFC (Alpha Innotech).

### Gelatin Gel Zymography for MMP Enzymatic Assessment

Gelatin gel zymography was performed to assess enzymatic activity of MMP-2 and MMP-9 in the EAE spinal cord tissue, as described previously [13]. Briefly, 25 μg of protein was separated by electrophoresis on 10% SDS-polyacrylamide gel, copolymerized with 1 mg/ml gelatin. Gelatinase activity was restored by renaturing proteins in 2.5% Triton X-100 followed by incubation with developing buffer for 48 h at 37° C. Gels were stained with Coomassie Blue for 30 min and de-stained (30% ethanol/10% acetic acid) until clear bands appeared as areas of gelatinase activity against a dark blue background. MMPs were identified based on their molecular weight and their density was measured.

### Multiplex Electrochemiluminescence Cytokine Assay

Levels of cytokines and chemokines in the spinal cord tissue of EAE mice were measured using the V-PLEX Mouse Cytokine 29-Plex Kit (Meso Scale Discovery, Rockville, MD) according to the manufacturer's instructions. Briefly, pre-coated plates were washed with PBS containing 0.05% Tween 20), and 40 lag of spinal cord tissue lysate with Diluent-41 was added to each well. The plates were sealed and incubated for 2 h at RT while shaking and washed three times. 25 μl of the corresponding sulfo-tagged detection antibodies was added to each well and incubated for 2 h. Finally, the plates were washed and 150 μl of Read buffer T (Meso-Scale) was added into each well, and the signal was measured immediately using a QuickPlex reader. Cytokine/chemokine levels were calculated with Discovery Workbench software (Meso-Scale) from calibration curves using four-parameter logistic fit.

### Enzyme Linked Immunosorbent Assay (ELISA) for Nrg-1β1 Detection

Mouse spinal cord tissue was homogenized in NP-40 lysis buffer containing protease inhibitor cocktail (Sigma). Mouse blood was collected with cardiac puncture in EDTA coated tubes. Samples were centrifuged at 2500 rpm for 25 min (4° C.). Blood plasma was collected and stored as aliquots at −80° C. until analysis. ELISA kit (DuoSet ELISA Development System; R&D Systems; DY377) was used to specifically detect Nrg-1β1 in blood plasma, spinal cord and spleen tissue lysates. Nrg-1β1 sandwich ELISA assay was performed according to the manufacturer's instructions, with standards (125-4000 μg/mL) and loading 25 μg of protein from each sample from spinal cord and spleen lysates. The Nrg-1β1 levels were calculated as pg/μg of tissue. For blood plasma analysis, direct ELISA was performed in the same manner, with the exception of omitting the first coating antibody. 50 μl of plasma samples was used for assay and results were expressed as pg/ml of plasma.

### Astrocytes, Microglia and Bone Marrow Derived Macrophage (BMDMs) In Vitro Studies

Astrocytes were cultured from cerebral cortex of C57bl/6 mice pups (P1-P3) by mechanically dissociation of the tissue and gradient filtration through 70lam and 40 μm cell strainer (BD Biosciences). Then, cells were seeded in complete DMEM media supplemented with 10%% fetal bovine serum (FBS, Gibco) and 1% penicillin—streptomycin-neomycin (PSN, Invitrogen). Upon reaching confluency at 3 weeks after culture, astrocytes were passaged into 6-well culture dishes for experimentation.

To isolate microglia, the cerebral cortex of C57bl/6 mice pups (P1-2) were dissected out and enzymatically dissociated in a solution containing papain (0.9 mg/rill; Worthington Biochemical), L-cysteine (0.2 mg/ml; Sigma) and EDTA (0.2 mg/ml; Sigma) diluted in HBSS (Invitrogen) for 45 minutes, at 37° C. Digested tissue was filtered through a 40 μm cell nylon strainer and seeded on poly-D-lysine (PDL) treated flasks in complete DMEM medium. The culture medium was refreshed every 3-4 days. Cultures were maintained at 37° C. and 5% CO₂. After 10 days, cultures were shaken upon reaching confluency for 3h at 250 rpm at 37° C. Medium was then filtered through 40 μm cell nylon strainer and centrifuged at 1000 rpm for 10 mins. Cells were seeded in PDL coated dishes in complete DMEM media (with 10% FBS).

BMDMs were harvested from 8-10-week-old C57bl/6 mice by flushing the femur and tibia, and the cells were seeded at a density of 107 cells per 100 mm Petri dish and grown in DMEM supplemented with 10% LADMAC (ATCC, CRL-2420) conditioned media, 10% FBS along with 2% penicillin-streptomycin for 1 week. BMDMs were passaged into 6-well dishes.

All three cells types were switched to serum free DMEM media after 24 h of seeding and treated with vehicle (0.1% BSA), Nrg-1β1 (50 or 200 ng/mL), LPS (100 ng/ml)+IFNγ (20 ng/ml) or Nrg-1β1+LPS+IFNγ for 72 h. Conditioned media was collected and stored at −80° C. until further use.

### Th1 and Th17 Polarization In Vitro

Naïve CD4+ T cells were purified from the spleens and lymph nodes of 10 weeks old female C57BL/6 mice using an EasySep Mouse naïve CD4+ T Cell Kit (19765, Stemcell Technologies). Isolated naïve CD4+ T cells were cultured in 24-well flat bottom plates (0.5×10⁶ cells per well) in 0.5 ml of complete RPMI 1640 media (supplemented with 10% Fetal bovine serum, 200 mM L-glutamine, 100U/ml penicillin/streptomycin and 5×10-5M 2-mercaptoethanol in the presence of 2 μg/ml plate-bound anti-mouse a-CD3 (17A2), 0.5 μg/ml soluble a-CD28 (37.51) and 50 ng/ml recombinant mouse IL-2 (all Shenandoah Biotehcnology, USA). Cells polarized to Th1 (5 ng/ml of recombinant IL-2, 10 ng/ml of recombinant IL-12 and 1 μg/ml of anti-IL-4), or Th17 (1 Kg/mL anti-IFN-γ, 1 μg/mL anti-IL-2, and 1 μg/mL anti-IL-4 antibodies, 20 ng/mL recombinant IL-6, 5 ng/ml recombinant IL-23 and 1 ng/mL TGF-β1. All recombinant cytokines were purchased from Shenandoah Biotechnology, USA and antibodies were purchased from eBioscience (Thermo Fisher, USA). Cells were expanded for 72h and transferred to fresh 24-well plates and cultured for another 48h under Th1 or Th17 polarizing conditions. Cells were washed and incubated in the presence of rhNrg-1-131 (50 and 200 ng/ml), 0.1% BSA (vehicle control), conditioned media from microglia, astrocytes or BMDMs. After 72 h, cells were incubated with 1 μl of Cell Stimulation Cocktail (plus protein transport inhibitors) (00-4975-03, eBioscience, ThermoFisher, USA) and added to each well for 5 hours. Viable cells were gated using Fixable Viability Stain 780 (565388, BD Biosciences). FACS data was acquired on the CytoFlex LX digital flow cytometry analyzer (Beckman Coulter, USA) and data was analyzed using FlowJo software.

### Proteomics Procedures and Analyses

### LC-MS/MS Sample Preparation and Analysis

Spinal cord lysates were digested, labelled and analysed by Manitoba Centre for Proteomics and Systems Biology (University of Manitoba) as per their standard procedures. Protein digests were performed as specified in the manufacturer's instructions for the Thermo Scientific's TMT10plex Isobaric Mass Tagging Kit (catalog #90110). TMT labeling was performed according to the manufacturer's instructions to label each biological replicate with a unique tag.

### Database for Annotation, Visualization, and Integrated Discovery (DAVID) Analysis

The database for annotation, visualization, and integrated discovery (DAVID) v6.8 is a comprehensive tool to perform functional annotation and understand biological meaning behind large list of genes associated with proteins. We performed a GO term enrichment analysis using DAVID and identified enriched biological themes and most relevant GO terms associated with our study. Only GO terms with an adjusted p value <0.05 were considered significant.

### CLUEGO Analysis

ClueGO plug-in of Cytoscape was used to generate protein pathways and to constitute the network of pathways based on the Gene Ontology. ClueGO enables to visualize the non-redundant biological terms for large clusters of genes in a functionally grouped network [28]. A ClueGO network reflects the relationships between the terms based on the similarity of their associated genes. Following parameters were used to perform ClueGo analysis: enrichment/depletion: two-sided hypergeometric statistical test; correction method: Benjamin-Hochberg; GO term range levels: 3-8; minimal number of genes for term selection: 5; minimal percentage of genes for term selection: 5%; K-score threshold: 0.8; general term selection method: smallest p value; group method: κ; minimal number of subgroups included in a group: 3; minimal percentage of shared genes between subgroups: 50%.

### Human Brain MS Specimens

Frozen post-mortem brain tissues were obtained from the United Kingdom Multiple Sclerosis Tissue Bank at Imperial College, London (provided by Richard Reynolds and Djordje Gveric). All MS tissues were obtained and used with approval from the institutional ethics committee of the University of Calgary. Six MS brain tissues with active lesions from individuals with chronic MS were assessed in this study. The lesions fulfilled the morphologic criteria of an active inflammatory demyelinating process consistent with MS when stained with H&E-LFB. Human tissue sections were fixed with 3.5% PFA before immunohistochemical staining.

### Human Plasma Samples

MS patients and normal participants were recruited at the Winnipeg Health Sciences Center, Winnipeg, Canada. All patients were diagnosed with MS according to the 2010 revised McDonald criteria. Based on the clinical diagnosis, plasma samples were categorised into different types/stages of MS-CIS, RRMS and SPMS. Healthy individuals served as controls. The study was approved by the the Health Research Ethics Board of the University of Manitoba. All participants gave written informed consent. Human blood was collected in sodium heparin tubes by standard venipuncture procedure. For blood plasma analysis, direct ELISA was performed in the same manner as described above for mouse plasma samples. All the stratifications undertaken with human plasma samples are representative of post-hoc analyses as these samples were repurposed from another unrelated clinical research project.

### Statistical Analysis

In all analyses, we performed unbiased assessments by utilizing randomization and blinding of methods. Using SigmaStat Software, Mann-Whitney U-test was used for human plasma data analysis. One-way ANOVA followed by Holm-Sidak post-hoc correction was used when comparing more than two groups. Two-way ANOVA was used for analysis of neurological scoring in EAE studies while Holm-Sidak post-hoc analyses was used when comparing mean of each time point. Mann-Whitney test was used while analysing EAE-based non-parametric data. Student's t-test was used when two groups were compared in EAE data. Specific statistical tests used for data analysis have been described in respective figure legends. The data were reported as means±standard error of the mean (SEM) unless specified otherwise and p<0.05 was considered statistically significant in all the analyses.

While the preferred embodiments of the invention have been described above, it will be recognized and understood that various modifications may be made therein, and the appended claims are intended to cover all such modifications which may fall within the spirit and scope of the invention.

### REFERENCES

1. Reich, D. S., C. F. Lucchinetti, and P. A. Calabresi, Multiple Sclerosis. N Engl J Med, 2018. 378(2): p. 169-180.
2. Faissner, S., et al., Progressive multiple sclerosis: from pathophysiology to therapeutic strategies. Nat Rev Drug Discov, 2019. 18(12): p. 905-922.
3. Weinshenker, B. G., et al., The natural history of multiple sclerosis: a geographically based study. I. Clinical course and disability. Brain, 1989. 112 (Pt 1): p. 133-46.
4. Miller, D. H., D. T. Chard, and O. Ciccarelli, Clinically isolated syndromes. Lancet Neural, 2012. 11(2): p. 157-69.
5. Kuhle, J., et al., Conversion from clinically isolated syndrome to multiple sclerosis: A large multicentre study. Mult Scler, 2015. 21(8): p. 1013-24.
6. Agrawal, S. M., et al., EMMPRIN: a novel regulator of leukocyte transmigration into the CNS in multiple sclerosis and experimental autoimmune encephalomyelitis. J Neurosci, 2011. 31(2): p. 669-77.
7. Dong, Y. and V. W. Yong, When encephalitogenic T cells collaborate with microglia in multiple sclerosis. Nat Rev Neurol, 2019. 15(12): p. 704-717.
8. Rawji, K. S. and V. W. Yong, The benefits and detriments of macrophages/microglia in models of multiple sclerosis. Clin Dev Immunol, 2013. 2013: p. 948976.
9. Yong, H. Y. F., et al., The benefits of neuroinflammation for the repair of the injured central nervous system. Cell Mol Immunol, 2019. 16(6): p. 540-546.
10. Baaklini, C. S., et al., Central Nervous System Remyelination: Roles of Glia and Innate Immune Cells. Front Mal Neurosci, 2019. 12: p. 225.
11. Kataria, H., A. Alizadeh, and S. Karimi-Abdolrezaee, Neuregulin-1/ErbB network: An emerging modulator of nervous system injury and repair. Prog Neurobiol, 2019. 180: p. 101643.
12. Kataria, H., et al., Neuregulin-1 promotes remyelination and fosters a pro-regenerative inflammatory response in focal demyelinating lesions of the spinal cord. Glia, 2018. 66(3): p. 538-561.
13. Alizadeh, A., et al., Neuregulin-1 positively modulates glial response and improves neurological recovery following traumatic spinal cord injury. Glia, 2017. 65(7): p. 1152-1175.
14. Alizadeh, A., et al., Neuregulin-1 elicits a regulatory immune response following traumatic spinal cord injury. J Neuroinflammation, 2018. 15(1): p. 53.
15. Shahriary, G. M., H. Kataria, and S. Karimi-Abdolrezaee, Neuregulin-1 Fosters Supportive Interactions between Microglia and Neural Stem/Progenitor Cells. Stem Cells Int, 2019. 2019: p. 8397158.
16. Li, Q., et al., Effect of neuregulin on apoptosis and expressions of STAT3 and GFAP in rats following cerebral ischemic reperfusion. J Mol Neurosci, 2009. 37(1): p. 67-73.
17. Li, Y., et al., Neuregulin-1 inhibits neuroinflammatory responses in a rat model of organophosphate-nerve agent-induced delayed neuronal injury. J Neuroinflammation, 2015. 12: p. 64.
18. Xu, Z., et al., Extended therapeutic window and functional recovery after intraarterial administration of neuregulin-1 after focal ischemic stroke. J Cereb Blood Flow Metab, 2006. 26(4): p. 527-35.
19. Mei, L. and W. C. Xiong, Neuregulin 1 in neural development, synaptic plasticity and schizophrenia. Nat Rev Neurosci, 2008. 9(6): p. 437-52.
20. Mei, L. and K. A. Nave, Neuregulin-ERBB signaling in the nervous system and neuropsychiatric diseases. Neuron, 2014. 83(1): p. 27-49.
21. Gauthier, M. K., et al., Dysregulation of the neuregulin-1-ErbB network modulates endogenous oligodendrocyte differentiation and preservation after spinal cord injury. Eur J Neurosci, 2013. 38(5): p. 2693-715.
22. Simmons, L. J., et al., Regulation of inflammatory responses by neuregulin-1 in brain ischemia and microglial cells in vitro involves the NF-kappa B pathway. J Neuroinflammation, 2016. 13(1): p. 237.
23. Viehover, A., et al., Neuregulin: an oligodendrocyte growth factor absent in active multiple sclerosis lesions. Dev Neurosci, 2001. 23(4-5): p. 377-86.
24. Tynyakov-Samra, E., et al., Reduced ErbB4 Expression in Immune Cells of Patients with Relapsing Remitting Multiple Sclerosis. Mult Scler Int, 2011. 2011: p. 561262.
25. Bahadori, Z., M. Behmanesh, and M. A. Sahraian, Two functional promoter polymorphisms of neuregulin 1 gene are associated with progressive forms of multiple sclerosis. J Neurol Sci, 2015. 351(1-2): p. 154-159.
26. Cannella, B., et al., The neuregulin, glial growth factor 2, diminishes autoimmune demyelination and enhances remyelination in a chronic relapsing model for multiple sclerosis. Proc Natl Acad Sci USA, 1998. 95(17): p. 10100-5.
27. Choi, B. Y., et al., Inhibition of NADPH oxidase activation reduces EAE-induced white matter damage in mice. J Neuroinflammation, 2015. 12: p. 104.
28. Bindea, G., et al., ClueGO: a Cytoscape plug-in to decipher functionally grouped gene ontology and pathway annotation networks. Bioinformatics, 2009. 25(8): p. 1091-3.
29. Tokita, Y., et al., Regulation of neuregulin expression in the injured rat brain and cultured astrocytes. J Neurosci, 2001. 21(4): p. 1257-64.
30. Kastin, A. J., V. Akerstrom, and W. Pan, Neuregulin-1-beta1 enters brain and spinal cord by receptor-mediated transport. J Neurochem, 2004. 88(4): p. 965-70.
31. Yong, V. W., et al., Metalloproteinases in biology and pathology of the nervous system. Nat Rev Neurosci, 2001. 2(7): p. 502-11.
32. Stephenson, E. L., et al., Chondroitin sulfate proteoglycans as novel drivers of leucocyte infiltration in multiple sclerosis. Brain, 2018. 141(4): p. 1094-1110.
33. Stephenson, E. L. and V. W. Yong, Pro-inflammatory roles of chondroitin sulfate proteoglycans in disorders of the central nervous system. Matrix Biol, 2018. 71-72: p. 432-442.
34. Stephenson, E. L., et al., Targeting the Chondroitin Sulfate Proteoglycans: Evaluating Fluorinated Glucosamines and Xylosides in Screens Pertinent to Multiple Sclerosis. ACS Cent Sci, 2019. 5(7): p. 1223-1234.
35. Ajami, B., et al., Infiltrating monocytes trigger EAE progression, but do not contribute to the resident microglia pool. Nat Neurosci, 2011. 14(9): p. 1142-9.
36. Jiang, Z., J. X. Jiang, and G. X. Zhang, Macrophages: a double-edged sword in experimental autoimmune encephalomyelitis. Immunol Lett, 2014. 160(1): p. 17-22.
37. Moline-Velazquez, V., et al., Myeloid cell distribution and activity in multiple sclerosis. Histol Histopathol, 2016. 31(4): p. 357-70.
38. Bizzozero, O. A., et al., Elevated protein carbonylation in the brain white matter and gray matter of patients with multiple sclerosis. J Neurosci Res, 2005. 81(5): p. 687-95.
39. Fischer, M. T., et al., Disease-specific molecular events in cortical multiple sclerosis lesions. Brain, 2013. 136(Pt 6): p. 1799-815.
40. Nikic, I., et al., A reversible form of axon damage in experimental autoimmune encephalomyelitis and multiple sclerosis. Nat Med, 2011. 17(4): p. 495-9.
41. Zamvil, S. S. and L. Steinman, The T lymphocyte in experimental allergic encephalomyelitis. Annu Rev Immunol, 1990. 8: p. 579-621.
42. Skundric, D. S., W. W. Cruikshank, and J. Drulovic, Role of IL-16 in CD4+ T cell-mediated regulation of relapsing multiple sclerosis. J Neuroinflammation, 2015. 12: p. 78.
43. Rostami, A. and B. Ciric, Role of Th17 cells in the pathogenesis of CNS inflammatory demyelination. J Neurol Sci, 2013. 333(1-2): p. 76-87.
44. Miron, V. E., et al., M2 microglia and macrophages drive oligodendrocyte differentiation during CNS remyelination. Nat Neurosci, 2013. 16(9): p. 1211-8.
45. Olah, M., et al., Identification of a microglia phenotype supportive of remyelination. Glia, 2012. 60(2): p. 306-21.
46. Brambilla, R., The contribution of astrocytes to the neuroinflammatory response in multiple sclerosis and experimental autoimmune encephalomyelitis. Acta Neuropathol, 2019. 137(5): p. 757-783.
47. Metz, L. M., et al., Trial of Minocycline in a Clinically Isolated Syndrome of Multiple Sclerosis. N Engl J Med, 2017. 376(22): p. 2122-2133.
48. Nave, K. A. and J. L. Salzer, Axonal regulation of myelination by neuregulin 1. Curr Opin Neurobiol, 2006. 16(5): p. 492-500.
49. Michailov, G. V., et al., Axonal neuregulin-1 regulates myelin sheath thickness. Science, 2004. 304(5671): p. 700-3.
50. Bartus, K., et al., Neuregulin-1 controls an endogenous repair mechanism after spinal cord injury. Brain, 2016. 139(Pt 5): p. 1394-416.
51. Ikawa, D., et al., Microglia-derived neuregulin expression in psychiatric disorders. Brain Behav Immun, 2017. 61: p. 375-385.
52. Loeb, J. A., E. T. Susanto, and G. D. Fischbach, The neuregulin precursor proARIA is processed to ARIA after expression on the cell surface by a protein kinase C-enhanced mechanism. Mol Cell Neurosci, 1998. 11(1-2): p. 77-91.
53. Loeb, J. A., et al., Expression patterns of transmembrane and released forms of neuregulin during spinal cord and neuromuscular synapse development. Development, 1999. 126(4): p. 781-91.
54. Pankonin, M. S., et al., Differential distribution of neuregulin in human brain and spinal fluid. Brain Res, 2009. 1258: p. 1-11.
55. Sosa, R. A., et al., The kinetics of myelin antigen uptake by myeloid cells in the central nervous system during experimental autoimmune encephalomyelitis. J Immunol, 2013. 191(12): p. 5848-57.
56. McMahon, E. J., et al., Epitope spreading initiates in the CNS in two mouse models of multiple sclerosis. Nat Med, 2005. 11(3): p. 335-9.
57. van Zwam, M., et al., Myelin ingestion alters macrophage antigen presenting function in vitro and in vivo. J Leukoc Biol, 2011. 90(1): p. 123-32.
58. Niimi, N., K. Kohyama, and Y. Matsumoto, Minocycline suppresses experimental autoimmune encephalomyelitis by increasing tissue inhibitors of metalloproteinases. Neuropathology, 2013. 33(6): p. 612-20.
59. Saederup, N., et al., Selective chemokine receptor usage by central nervous system myeloid cells in CCR2-red fluorescent protein knock-in mice. PLoS One, 2010. 5(10): p. e13693.
60. Qin, H., et al., SOCS3 deficiency promotes M1 macrophage polarization and inflammation. J Immunol, 2012. 189(7): p. 3439-48.
61. Dyck, S., et al., Perturbing chondroitin sulfate proteoglycan signaling through LAR and PTPsigma receptors promotes a beneficial inflammatory response following spinal cord injury. J Neuroinflammation, 2018. 15(1): p. 90.
62. Nuttall, R. K., et al., Metalloproteinases are enriched in microglia compared with leukocytes and they regulate cytokine levels in activated microglia. Glia, 2007. 55(5): p. 516-26.
63. Larochelle, C., J. I. Alvarez, and A. Prat, How do immune cells overcome the blood-brain barrier in multiple sclerosis? FEBS Lett, 2011. 585(23): p. 3770-80.
64. McManus, C., et al., MCP-1, MCP-2 and MCP-3 expression in multiple sclerosis lesions: an immunohistochemical and in situ hybridization study. J Neuroimmunol, 1998. 86(1): p. 20-9.
65. Gerwien, H., et al., Imaging matrix metalloproteinase activity in multiple sclerosis as a specific marker of leukocyte penetration of the blood-brain barrier. Sci Transl Med, 2016. 8(364): p. 364ra152.
66. Lok, J., et al., Neuregulin-1 effects on endothelial and blood-brain-barrier permeability after experimental injury. Transl Stroke Res, 2012. 3 Suppl 1: p. S119-24.
67. Asher, R. A., et al., Neurocan is upregulated in injured brain and in cytokine-treated astrocytes. J Neurosci, 2000. 20(7): p. 2427-38.
68. Hallmann, R., et al., The regulation of immune cell trafficking by the extracellular matrix. Curr Opin Cell Biol, 2015. 36: p. 54-61.
69. Properzi, F., et al., Chondroitin 6-sulphate synthesis is up-regulated in injured CNS, induced by injury-related cytokines and enhanced in axon-growth inhibitory glia. Eur J Neurosci, 2005. 21(2): p. 378-90.
70. Plemel, J. R., et al., Microglia response following acute demyelination is heterogeneous and limits infiltrating macrophage dispersion. Sci Adv, 2020. 6(3): p. eaay6324.
71. Furlan, R., et al., Intrathecal delivery of IFN-gamma protects C57BL/6 mice from chronic-progressive experimental autoimmune encephalomyelitis by increasing apoptosis of central nervous system-infiltrating lymphocytes. J Immunol, 2001. 167(3): p. 1821-9.
72. Wen, L., et al., Neuregulin 1 regulates pyramidal neuron activity via ErbB4 in parvalbumin-positive interneurons. Proc Natl Acad Sci USA, 2010. 107(3): p. 1211-6.
73. Jones, A. and D. Hawiger, Peripherally Induced Regulatory T Cells: Recruited Protectors of the Central Nervous System against Autoimmune Neuroinflammation. Front Immunol, 2017. 8: p. 532.
74. Dombrowski, Y., et al., Regulatory T cells promote myelin regeneration in the central nervous system. Nat Neurosci, 2017. 20(5): p. 674-680.
75. Koutrolos, M., et al., Treg cells mediate recovery from EAE by controlling effector T cell proliferation and motility in the CNS. Acta Neuropathol Commun, 2014. 2: p. 163.
76. Marballi, K., et al., In vivo and in vitro genetic evidence of involvement of neuregulin 1 in immune system dysregulation. J Mol Med (Bed), 2010. 88(11): p. 1133-41.
77. Mills, E. A. and Y. Mao-Draayer, Understanding Progressive Multifocal Leukoencephalopathy Risk in Multiple Sclerosis Patients Treated with Immunomodulatory Therapies: A Bird's Eye View. Front Immunol, 2018. 9: p. 138.

1. A method of treating or prophylactically treating multiple sclerosis comprising:
administering to an individual in need of such treatment an effective amount of Nrg-1β1.

2. The method according to claim 1 wherein the Nrg-1β1 is administered on a dosage regimen or schedule.

3. The method according to claim 1 wherein the effective amount of Nrg-1β1restores reduced levels of Nrg-1 in the blood and/or in the CNS.

4. The method according to claim 1 wherein the individual in need of such treatment is an individual who has early stage multiple sclerosis, severe multiple sclerosis, delayed multiple sclerosis or has been diagnosed with multiple sclerosis but is in remission.

5. The method according to claim 1 wherein the individual who is in need of such treatment is a pre-symptomatic individual who is at risk of developing MS.

6. The method according to claim 1 wherein the individual in need of such treatment is an individual who has a level of Nrg-1β1 that is below a threshold level of Nrg-1β1.

7. The method according to claim 6 wherein the threshold level of Nrg-1β1 is determined from a healthy individual.

8. The method according to claim 7 wherein the threshold level of Nrg-1β1 is about 50% or that of a corresponding healthy individual.

9. The method according to claim 1 wherein the effective amount is about 0.25 to about 10 pg Nrg-1β1 per kg body weight of the individual.

10. The method according to claim 1 wherein the Nrg-1β1 is co-administered with a second medicament for treating multiple sclerosis.

11. The method according to claim 10 wherein the second medicament for treating multiple sclerosis is selected from the group consisting of: interferon β1, glatiramer acetate, fingolimod, and alemtuzumab.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)
